(19) United States
(12) Patent Application Publication
(10) Pub. No.: US 2007/0178166 A1
(43) Pub. Date: Aug. 2, 2007

(54) PROCESSES FOR MAKING PARTICLE-BASED PHARMACEUTICAL FORMULATIONS FOR PULMONARY OR NASAL ADMINISTRATION

(75) Inventors: Howard Bernstein, Cambridge, MA (US); Shaina Brito, Winchester, MA (US); Donald E. Chickering III, Framingham, MA (US); Eric K. Huang, Cambridge, MA (US); Rajeev A. Jain, Framingham, MA (US); Julie A. Straub, Winchester, MA (US)

Correspondence Address: SUTHERLAND ASBILL & BRENNAN LLP, 999 PEACHTREE STREET, N.E., ATLANTA, GA, 30309, US

(73) Assignee: ACUSPHERE, INC., Watertown, MA (US)

(21) Appl. No.: 11/610,814

(22) Filed: Dec. 14, 2006

Related U.S. Application Data
(60) Provisional application No. 60/750,462, filed on Dec. 15, 2005.

(51) Int. Cl. A61K 9/14

(52) U.S. Cl. 424/499; 424/489; 424/502

(57) ABSTRACT

Dry powder pharmaceutical formulations for pulmonary or nasal administration are made to provide an improved respired dose. These formulations may be blends of milled blends and may include a phospholipid, alone or in combination with other excipient materials. In one case, the process includes the steps of (a) providing particles which comprise a pharmaceutical agent, (b) blending the particles with particles of at least one first excipient to form a first powder blend; (c) milling the first powder blend to form a milled blend which comprises microparticles or nanoparticles of the pharmaceutical agent; and (d) blending the milled blend with particles of a second excipient to form a blended dry powder blend pharmaceutical formulation suitable for pulmonary or nasal administration.

### CROSS-REFERENCE TO RELATED APPLICATIONS

This application claims benefit of U.S. Provisional Application No. 60/750,462, filed Dec. 15, 2005. The application is incorporated herein by reference.

### BACKGROUND OF THE INVENTION

This invention is generally in the field of pharmaceutical compositions comprising particles, such as microparticles, and more particularly to methods for making particulate blend formulations for pulmonary or nasal administration.

Delivery of pharmaceutical agents to the lungs and through the lungs to the body represents a significant medical opportunity. Many pulmonary or nasal drug formulations desirably are produced in a dry powder form. Pulmonary dosage forms of therapeutic microparticles require that the microparticles are dispersed in a gas, typically air, and then inhaled into the lungs where the particles dissolve/release the therapeutic agent. Similarly, nasal dosage forms also require that the microparticles be dispersed in a gas, typically air, and then inhaled into the nasal cavity, where the particles dissolve/release the therapeutic agent. It is important that the drug-containing particles disperse well during pulmonary or nasal administration.

In pulmonary formulations, pharmaceutical agent particles are often combined with one or more excipient materials, at least in part, to improve dispersibility of the drug particles. In addition, excipients often are added to the microparticles and pharmaceutical agents in order to provide the microparticle formulations with other desirable properties or to enhance processing of the microparticle formulations. For example, the excipients can facilitate administration of the microparticles, minimize microparticle agglomeration upon storage or upon reconstitution, facilitate appropriate release or retention of the active agent, and/or enhance shelf life of the product. It is also important that the process of combining these excipients and microparticles yield a uniform blend. Combining these excipients with the microparticles can complicate production and scale-up; it is not a trivial matter to make such microparticle pharmaceutical formulations, particularly on a commercial scale.

How much of the drug particles that actually are delivered into the lungs when a dose is inhaled typically is referred to as the respired dose. The respired dose depends on many factors, including the dispersibility of the blend of drug particles and excipient particles. It would therefore be useful to provide a manufacturing process that creates well dispersing microparticle formulations and thus increased respirable doses.

Furthermore, certain desirable excipient materials are difficult to mill or blend with pharmaceutical agent microparticles. For example, excipients characterized as liquid, waxy, non-crystalline, or non-friable are not readily blended uniformly with drug containing particles. Conventional dry blending of such materials may not yield the uniform, intimate mixtures of the components, which pharmaceutical formulations require. For example, dry powder formulations therefore should not be susceptible to batch-to-batch or intra-batch compositional variations. Rather, production processes for a pharmaceutical formulation must yield consistent and accurate dosage forms. Such consistency in a dry powder formulation may be difficult to achieve with an excipient that is not readily blended or milled. It therefore would be desirable to provide methods for making uniform blends of microparticles and difficult to blend excipients. Such methods desirably would be adaptable for efficient, commercial scale production.

It therefore would be desirable to provide improved methods for making blended particle or microparticle pharmaceutical formulations that have high content uniformity and that disperse well upon pulmonary or nasal administration.

### SUMMARY OF THE INVENTION

Methods are provided for making a dry powder pharmaceutical formulation for pulmonary or nasal administration. In one embodiment, the method includes the steps of (a) providing particles which comprise a pharmaceutical agent; (b) blending the particles with particles of at least one first excipient to form a first powder blend; (c) milling the first powder blend to form a milled blend which comprises microparticles or nanoparticles of the pharmaceutical agent; and (d) blending the milled blend with particles of a second excipient to form a blended dry powder blend pharmaceutical formulation suitable for pulmonary or nasal administration, wherein the particles of second excipient are larger than the microparticles or nanoparticles in the milled blend and the second excipient is selected from the group consisting of sugars, sugar alcohols, starches, amino acids, and combinations thereof. In another aspect, a method is provided for making a dry powder pharmaceutical formulation for pulmonary or nasal administration having improved stability, comprising the steps of: (a) providing first particles which comprise a pharmaceutical agent (which may be thermally labile) and may further include a shell material; (b) blending the first particles with second particles of at least one excipient to form a powder blend; and (c) milling the powder blend to form a powder blend pharmaceutical formulation suitable for pulmonary or nasal administration, wherein the powder blend comprises microparticles which comprise the pharmaceutical agent, wherein the pharmaceutical agent, or the microparticles, in the powder blend pharmaceutical formulation of step (c) have greater stability at storage conditions than the particles of step (a) or the powder blend of step (b). In various embodiments, the milling step in the foregoing methods comprises jet milling.

In one embodiment of the foregoing methods, the particles of the at least one first excipient comprise a material selected from sugars, sugar alcohols, starches, amino acids, and combinations thereof. In various embodiments, the particles of the first excipient, the second excipient, or both, may be lactose. In one embodiment, the particles of step (a) are microparticles. The particles of step (a) may be made by a spray drying process. Optionally, the particles of step (a) may further include a shell material, such as a biocompatible synthetic polymer. In one embodiment, the microparticles of the milled blend that comprise the pharmaceutical agent have a volume average diameter of between 1 and 10 μm. In one embodiment, the particles of the second excipient have a volume average diameter between 20 and 500 μm. Examples of pharmaceutical agents that may be used in the present methods and pulmonary or nasal formulations include budesonide, fluticasone propionate, beclomethasone dipropionate, mometasone, flunisolide, triamcinolone acetonide, albuterol, formoterol, salmeterol, cromolyn sodium, ipratropium bromide, testosterone, progesterone, estradiol, enoxaprin, ondansetron, sumatriptan, sildenofil, dornase alpha, iloprost, heparin, low molecular weight heparin, desirudin, or a combination thereof.

In another aspect, a method is provided for making a dry powder pharmaceutical formulation for pulmonary or nasal administration that includes the steps of (a) providing particles which comprise a pharmaceutical agent; (b) blending the particles with particles of a pre-processed excipient to form a primary blend, wherein the pre-processed excipient is prepared by (i) dissolving a bulking agent and at least one non-friable excipient in a solvent to form an excipient solution, and (ii) removing the solvent from the excipient solution to form the pre-processed excipient in dry powder form; and (c) milling the primary blend to form a milled pharmaceutical formulation blend suitable for pulmonary or nasal administration. Optionally, one may include, as a step (d), blending the milled pharmaceutical formulation blend with particles of a second excipient to form a blended dry powder blend pharmaceutical formulation suitable for pulmonary or nasal administration. The step of removing the solvent may include spray drying, lyophilization, vacuum drying, or freeze drying. In one embodiment, the particles of second excipient are larger than the microparticles or nanoparticles in the milled blend and the second excipient is selected from the group consisting of sugars, sugar alcohols, starches, amino acids, and combinations thereof. In one embodiment, the bulking agent comprises at least one sugar, sugar alcohol, starch, amino acid, or combination thereof. Examples of bulking agents include lactose, sucrose, maltose, mannitol, sorbitol, trehalose, galactose, xylitol, eryihritol, and combinations thereof. The non-friable excipient may be a liquid, waxy, or non-crystalline compound. In one embodiment, the non-friable excipient comprises a surfactant, particularly a waxy or liquid surfactant. In one embodiment, the pre-processed excipient comprises a combination of lactose and a phospholipid or a fatty acid. The dry powder blend pharmaceutical formulation may be thermally-labile.

In another aspect, a method is provided for making a dry powder blend pharmaceutical formulation that includes the steps of: (a) providing microparticles which comprise a pharmaceutical agent; (b) blending the microparticles with particles of at least one first excipient to form a first powder blend; (c) milling the first powder blend to form a milled blend; and (d) blending the milled blend with particles of a second excipient, wherein the particles of second excipient are larger than the microparticles in the milled blend, to form a blended dry powder blend pharmaceutical formulation, wherein the blended dry powder blend pharmaceutical formulation from step (d) exhibits an increased respirable dose as compared to a respirable dose of the microparticles of step (a), the first powder blend of step (b), or the milled blend of step (c). In one embodiment, the milling of step (c) includes jet milling. In one embodiment, the second excipient is selected from sugars, sugar alcohols, starches, amino acids, and combinations thereof. In one embodiment, the microparticles of the milled blend which comprise the pharmaceutical agent have a volume average diameter of between 1 and 10 μm. In another embodiment, the particles of the second excipient have a volume average diameter between 20 and 500 μm.

In another aspect, pharmaceutical formulations made by any of the foregoing methods are provided. In one embodiment, a dry powder pulmonary or nasal formulation is provided that includes a blend of a milled blend of (i) microparticles which comprise a pharmaceutical agent, and (ii) excipient particles; and particles of a sugar or sugar alcohol, which particles are larger than the microparticles or excipient particles of the milled blend, wherein the blend which exhibits an increased respirable dose as compared to a respirable dose of combinations of the microparticles, the excipient particles, and the particles of sugar or sugar alcohol which are not blend-of-milled-blend combinations. Examples of pharmaceutical agents include budesonide, fluticasone propionate, beclomethasone dipropionate, mometasone, flunisolide, triamcinolone acetonide, albuterol, formoterol, salmeterol, cromolyn sodium, ipratropium bromide, testosterone, progesterone, estradiol, enoxaprin, ondansetron, sumatriptan, sildenofilt, domase alpha, iloprost, heparin, low molecular weight heparin, desirudin, or a combination thereof. In one embodiment, the pharmaceutical agent has a solubility in water of less than 10 mg/mL at 25° C. In one embodiment, the excipient particles comprise a sugar, a sugar alcohol, a starch, an amino acid, or a combination thereof. In one embodiment, the sugar or sugar alcohol comprises lactose, sucrose, maltose, mannitol, sorbitol, trehalose, galactose, xylitol, erythritol, or a combination thereof. In one case, both the excipient particles and the particles of the sugar or sugar alcohol comprise lactose. In one embodiment, the microparticles which include pharmaceutical agent have a volume average diameter of less than 10 μm. For example, the pharmaceutical agent microparticles may have a volume average diameter of less than 5 μm. Optionally, the particles of step (a) may further include a shell material, such a biocompatible synthetic polymer. In one embodiment, the particles of the sugar or sugar alcohol have a volume average diameter between 20 and 500 μm.

In another aspect, a dry powder pharmaceutical formulation for pulmonary or nasal administration is provided which includes a blend of at least one phospholipid, such as dipalmitoyl phosphatidylcholine, and particles of a pharmaceutical agent. The phospholipid may be blended with the pharmaceutical agent before or after milling. In one embodiment, the formulation may be in the form of a blend of a milled blend. For instance, the formulation may comprise a milled blend made by (a) providing particles which comprise a pharmaceutical agent; (b) blending the particles with at least one phospholipid and tertiary excipient particles to make a first powder blend; (c) milling the first powder blend to form a milled blend which comprises microparticles or nanoparticles of the pharmaceutical agent, the at least one phospholipid, and tertiary excipient particles; and (d) blending the milled blend with particles of a sugar or sugar alcohol, which particles are larger than the microparticles (or nanoparticles) or excipient particles of the milled blend. The at least one phospholipid may include dipalmitoyl phosphatidylcholine.

### BRIEF DESCRIPTION OF THE DRAWINGS

FIG. 1 is a process flow diagram of one embodiment of a process for making a pulmonary or nasal dosage form of a pharmaceutical formulation which includes a dry powder blend of an excipient and a milled blend of a drug and another excipient as described herein.

FIG. 2 is a process flow diagram of one embodiment of a process for making a pulmonary or nasal dosage form of a pharmaceutical formulation which includes a milled dry powder blend of a drug and a pre-processed excipient as described herein.

FIG. 3 is a process flow diagram of one embodiment of a process for pre-processing a non-friable excipient into a dry powder form.

FIGS. 4A-B are light microscope images of reconstituted celecoxib from a blend of excipient particles and celecoxib particles.

FIGS. 5A-B are light microscope images of reconstituted celecoxib from a blend of excipient particles and milled celecoxib particles.

FIGS. 6A-B are light microscope images of reconstituted celecoxib from a jet milled blend of excipient particles and celecoxib particles.

### DETAILED DESCRIPTION OF THE INVENTION

Improved processing methods have been developed for making a pulmonary or nasal dosage form of a pharmaceutical formulation that includes a highly uniform blend of pharmaceutical agent particles and excipient particles, and better stability of dry powder formulations under storage conditions. It has been determined that better dispersibility of such formulations may be obtained by the ordered steps of blending particles of pharmaceutical agent with an excipient, milling the resulting blend, and then blending additional excipient particles with the first blend, as compared to blends prepared without this combination of steps. It has also been beneficially discovered that certain useful but difficult-to-mill (or difficult-to-blend) excipient materials can be used in the process if they are themselves first subjected to a “pre-processing” treatment that transforms the liquid, waxy, or otherwise non-friable excipient into a dry powder form that is suitable for blending and milling in a dry powder form. By blending a milled blend, it was found that the dry powder blend advantageously exhibited a better respirable dose of the pharmaceutical agent, which is believed to be due to uniformity of the blends with two different sized excipient particles to aid in dispersibility and particle flight. Thus, delivery of the pharmaceutical agent to the lungs or nasal cavity is improved with blend formulations made by the presently described processes.

In another aspect, an improved respirable dose beneficially can be attained by incorporating at least one phospholipid into the dry powder pharmaceutical formulation. Studies show that pulmonary formulations comprising a milled blend of dipalmitoyl phosphatidylcholine (DPPC) and particles of a therapeutic agent have improved respirable dose relative to comparable formulations made without DPPC, with the highest respirable doses observed for blends of jet milled blends with DPPC in the initial blend before milling.

As used herein, the term “dispersibility” includes the suspendability of a powder (e.g., a quantity or dose of microparticles) within a gas (e.g., air) as well as the dispersibility of the powder within an aqueous liquid environment, as in contact with fluids in the lungs or in a liquid carrier for nebulization. Accordingly, the term “improved dispersibility” refers to a reduction of particle-particle interactions of the microparticles of a powder within a gas, leading to increased respirable dose, which can be evaluated using methods that examine the increase in concentration of suspended particles or a decrease in agglomerates. These methods include visual evaluation for turbidity of the suspension, direct turbidity analysis using a turbidimeter or a visible spectrophotometer, light microscopy for evaluation of concentration of suspended particles and/or concentration of agglomerated particles, or Coulter counter analysis for particle concentration in suspension. Improvements in dispersibility can also be assessed as an increase in wettability of the powder using contact angle measurements. Improvements in dispersity within air can be evaluated using methods such as cascade impaction, liquid impinger analysis, time of flight methods (such as an Aerosizer, TSI), and plume geometry analysis.

The pharmaceutical formulations made as described herein are intended to be administered to a patient (i.e., human or animal in need of the pharmaceutical agent) to deliver an effective amount of a therapeutic, diagnostic, or prophylactic agent. For example, the blend formulations can be delivered by oral inhalation to the lungs using a dry powder inhaler or metered dose inhaler known in the art.

Advantageously, the methods described herein may provide improved storage stability of the pharmaceutical product. Accordingly, the processing methods are believed to be particularly suitable for producing blends comprising microparticles containing thermally labile pharmaceutical agents, such as many proteins and polypeptides. As used herein, the term “thermally labile” refers to substances, such as biologically active agents that lose a substantial amount of activity or polymers that physically degrade, when warmed to elevated temperatures, such as temperatures greater than physiological temperatures, e.g., about 37° C.

As used herein, the terms “comprise,” “comprising,” “include,” and “including” are intended to be open, non-limiting terms, unless the contrary is expressly indicated.

The Methods

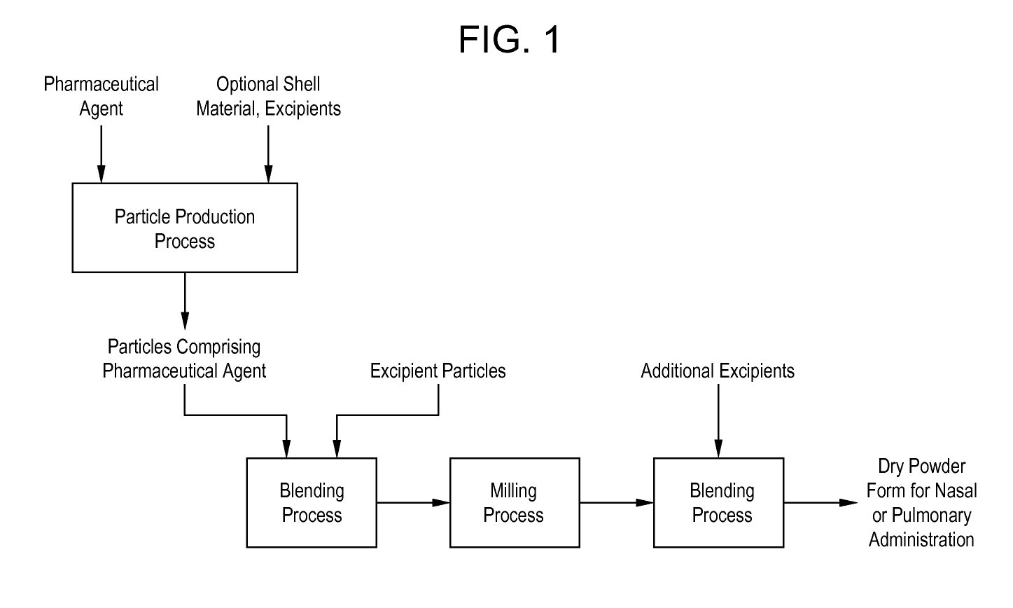

In one aspect, it has been found advantageous to make a dry powder pharmaceutical formulation for pulmonary or nasal administration by a process that includes making a blend from a first blend that has been subjected to a milling process. It has been discovered that the process of production is a key to making better dry powder blends, and this process may provide a comparatively better respirable dose of pharmaceutical agent. In one embodiment, the method for making a dry powder pharmaceutical formulation for pulmonary or nasal administration comprises the steps of: (a) providing particles which comprise a pharmaceutical agent; (b) blending the particles with particles of at least one first excipient to form a first powder blend; (c) milling the first powder blend to form a milled blend which comprises microparticles or nanoparticles of the pharmaceutical agent; and (d) blending the milled blend with particles of a second excipient to form a blended dry powder blend (a blended milled blend) pharmaceutical formulation suitable for pulmonary or nasal administration. See FIG. 1. In a preferred embodiment, the particles of second excipient preferably are larger than the microparticles or nanoparticles in the milled blend and the second excipient preferably is selected from sugars, sugar alcohols, starches, amino acids, and combinations thereof. In another preferred embodiment, the blended powder blend pharmaceutical formulation from step (d) exhibits an increased respirable dose as compared to a respirable dose of the microparticles of step (a), the first powder blend of step (b), or the milled blend of step (c). In one embodiment, the particles of the at least one first excipient comprise a material selected from sugars, sugar alcohols, starches, amino acids, and combinations thereof. In one example, the particles of second excipient comprise lactose. In another example, the particles of at least one first excipient and the particles of the second excipient both comprise lactose. In one embodiment, the particles of step (a) are microparticles. n a preferred embodiment, the milling comprises jet milling. In one embodiment, the particles of step (a) are made by a spray drying process.

In another aspect, a method is provided for making a dry powder pharmaceutical blend formulation for pulmonary or nasal administration having improved stability. Again, it has been discovered that the process of production is a key to making better dry powder blends, and this process may provide comparatively better stability of the pharmaceutical agent or microparticles comprising the pharmaceutical agent or agents, particularly thermally labile pharmaceutical agents. In one embodiment, the method comprises the steps of: (a) providing first particles which comprise a pharmaceutical agent; (b) blending the first particles with second particles of at least one excipient to form a powder blend; and (c) milling the powder blend to form a powder blend pharmaceutical formulation suitable for pulmonary or nasal administration, wherein the pharmaceutical agent, or microparticles comprising the pharmaceutical agent, has greater stability at storage conditions in the powder blend pharmaceutical formulation of step (c) than the particles of step (a) or in the powder blend of step (b). Examples show improved stability at storage conditions for material in an open container and material in closed containers

As used herein, the phrase “stability at storage conditions” refers to how the quality of the dry powder blend product varies with time under the influence of temperature, humidity, and other environmental factors, which is indicative of the degree of degradation or decomposition of the product that may be expected to occur during shipment and storage of the product. Stability testing standards are known in the art, and guidelines relevant thereto are provided by U.S. Food and Drug Administration (FDA). The particular testing parameters selected may vary depending upon the particular pharmaceutical agent or product being assessed. Examples of conditions at which stability may be assessed include 40±2° C./75±5% RH and 30±2° C./60±5% RH.

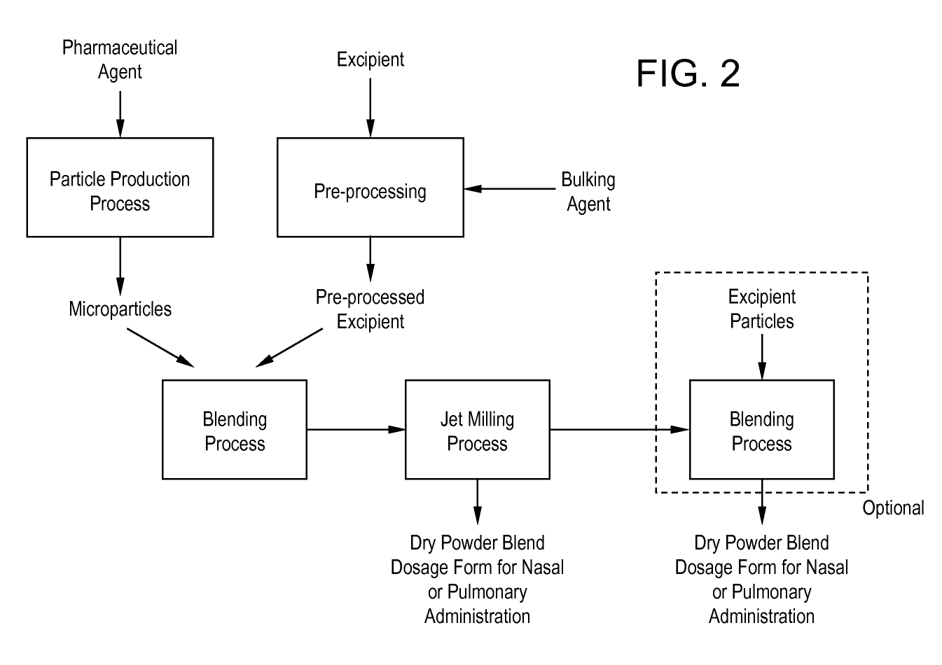

In one embodiment, a method is provided for making a dry powder pharmaceutical formulation for pulmonary or nasal administration, which includes the steps of: (a) providing particles which comprise a pharmaceutical agent; (b) blending the particles with particles of a pre-processed excipient to form a primary blend, wherein the pre-processed excipient is prepared by (i) dissolving a bulking agent and at least one non-friable excipient in a solvent to form an excipient solution, and (ii) removing the solvent from the excipient solution to form the pre-processed excipient in dry powder form; and (c) milling the primary blend to form a milled pharmaceutical formulation blend suitable for pulmonary or nasal administration. See FIG. 2 (without optional step). In one example, the step of removing the solvent comprises spray drying. In another example, the step of removing the solvent comprises lyophilization, vacuum drying, or freeze drying. In preferred embodiments, the bulking agent includes at least one sugar, sugar alcohol, starch, amino acid, or combination thereof. For example, the bulking agent may be selected from lactose, sucrose, maltose, mannitol, sorbitol, trehalose, galactose, xylitol, erythritol, and combinations thereof. In one embodiment, the non-friable excipient includes a liquid, waxy, or non-crystalline compound. In one embodiment, the non-friable excipient comprises a surfactant, such as a waxy or liquid surfactant. In one embodiment, the preprocessed excipient comprises a combination of lactose and a phospholipid or a fatty acid. In one embodiment, the pharmaceutical agent is thermally-labile.

In one embodiment, the method further comprises (d) blending the milled pharmaceutical formulation blend with particles of a second excipient to form a blended dry powder blend pharmaceutical formulation suitable for pulmonary or nasal administration. The particles of second excipient preferably may be larger than the microparticles or nanoparticles in the milled blend and the second excipient preferably is selected from sugars, sugar alcohols, starches, amino acids, and combinations thereof. See FIG. 2 (with optional step).

In one embodiment, a phospholipid is blended with the pharmaceutical agent to be administered. The phospholipid can be combined with the pharmaceutical agent before or after milling. In one embodiment, the formulation may be in the form of a blend of a milled blend. For instance, the formulation may comprise a milled blend made by (a) providing particles which comprise a pharmaceutical agent; (b) blending the particles with at least one phospholipid and tertiary excipient particles to make a first powder blend; (c) milling the first powder blend to form a milled blend which comprises microparticles or nanoparticles of the pharmaceutical agent, the at least one phospholipid, and tertiary excipient particles; and (d) blending the milled blend with particles of a sugar or sugar alcohol, where the sugar or sugar alcohol particles are larger than the microparticles or excipient particles of the milled blend. In another embodiment, the phospholipid may be milled and then added to, or blended with. a pharmaceutical composition for pulmonary or nasal delivery.

Phospholipids that may be used include phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phosphatidylcholines include such as dioleoylphosphatidylcholine, dimyristoylphosphatidylcholine (DMPC), dipentadecanoylphosphatidylcholine dilauroylphosphatidylcholine, dipaimitoylphosphatidylcholine (DPPC), distearoylphosphatidylcholine (DSPC), diarachidoylphosphatidylcholine (DAPC), dibehenoylphosphatidylcholine (DBPC), ditricosanloylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used. Examples of phosphatidylethanolamines include dicaprylphosphatidylethanolamine, dioctanoylphosphatidylethanolamine, dilauroylphosphatidylethanolamine, dimyristoylphosphatidylethanolamine (DMPE), dipalmitoylphosphatidylethanolamine (DPPE), dipalmitoleoylphosphatidylethanolaminie, distearoylphosphatidylethanolamine (DSPE), dioleoylphosphatidylethanolamine, and dilineoylphosphatidylethanolamine. Examples of phosphatidylglycerols include dicaprylphosphatidylglycerol, dioctanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglycerol, and dilineoylphosphatidylglycerol. Preferred phospholipids include DMPC, DPPC, DAPC, DSPC, DTPC, DBPC, DLPC, DMPG, DPPG, DSPG, DMPE, DPPE, and DSPE, and most preferably DPPC, DAPC and DSPC.

The processes described herein generally can be conducted using batch, continuous, or semi-batch methods. These processes described herein optionally may further include separately milling some or all of the components (e.g., pharmaceutical agent particles, excipient particles) of the blended formulation before they are blended together. In preferred embodiments, the excipients and pharmaceutical agent are in a dry powder form.

Particle Production

The skilled artisan can envision many ways of making particles useful for the methods and formulations described herein, and the following examples describing how particles may be formed or provided are not intended to limit in any way the methods and formulations described and claimed herein. The particles comprising pharmaceutical agent that are used or included in the methods and formulations described herein can be made using a variety of techniques known in the art. Suitable techniques may include solvent precipitation, crystallization, spray drying, melt extrusion. compression molding, fluid bed drying, solvent extraction, hot melt encapsulation, phase inversion encapsulation, and solvent evaporation.

For instance, the microparticles may be produced by crystallization. Methods of crystallization include crystal formation upon evaporation of a saturated solution of the pharmaceutical agent, cooling of a hot saturated solution of the pharmaceutical agent, addition of antisolvent to a solution of the pharmaceutical agent (drowning or solvent precipitation), pressurization, addition of a nucleation agent such as a crystal to a saturated solution of the pharmaceutical agent, and contact crystallization (nucleation initiated by contact between the solution of the pharmaceutical agent and another item such as a blade).

Another way to form the particles, preferably microparticles, is by spray drying. See, e.g., U.S. Pat. No. 5,853,698 to Straub et al.; U.S. Pat. No. 5,611,344 to Bernstein et al.; U.S. Pat. No. 6,395,300 to Straub et al.; and U.S. Pat. No. 6,223,455 to Chickering III et al., which are incorporated herein by reference. As defined herein, the process of “spray drying” a solution containing a pharmaceutical agent and/or shell material refers to a process wherein the solution is atomized to form a fine mist and dried by direct contact with hot carrier gases. Using spray drying equipment available in the art, the solution containing the pharmaceutical agent and/or shell material may be atomized into a drying chamber, dried within the chamber, and then collected via a cyclone at the outlet of the chamber. Representative examples of types of suitable atomization devices include ultrasonic, pressure feed, air atomizing, and rotating disk. The temperature may be varied depending on the solvent or materials used. The temperature of the inlet and outlet ports can be controlled to produce the desired products. The size of the particulates of pharmaceutical agent and/or shell material is a function of the nozzle used to spray the solution of pharmaceutical agent and/or shell material, nozzle pressure, the solution and atomization flow rates, the pharmaceutical agent and/or shell material used, the concentration of the pharmaceutical agent and/or shell material, the type of solvent, the temperature of spraying (both inlet and outlet temperature), and the molecular weight of a shell material such as a polymer or other matrix material.

A further way to make the particles is through the use of solvent evaporation, such as described by Mathiowitz et al., J. Scanning Microscopy, 4:329 (1990); Beck et al. Fertil. Steril, 31:545 (1979) and Benita et al., J. Pharm. Sci., 73:1721 (1984). In still another example, hot-melt microencapsulation may be used, such as described in Mathiowitz et al., Reactive Polymers, 6:275 (1987). In another example, a phase inversion encapsulation may be used, such as described in U.S. Pat. No. 6,143,211 to Mathiowitz et al. This causes a phase inversion and spontaneous formation of discrete microparticles, typically having an average particle size of between 10 nm and 10 μm.

In yet another approach, a solvent removal technique may be used, wherein a solid or liquid pharmaceutical agent is dispersed or dissolved in a solution of a shell material in a volatile organic solvent and the mixture is suspended by stirring in an organic oil to form an emulsion. Unlike solvent evaporation, however, this method can be used to make microparticles from shell materials such as polymers with high melting points and different molecular weights. The external morphology of particles produced with this technique is highly dependent on the type of shell material used.

In another approach, an extrusion technique may be used to make microparticles of shell materials, For example, such microparticles may be produced by dissolving the shell material (e.g., gel-type polymers, such as polyphosphazene or polymethylmethacrylate) in an aqueous solution, homogenizing the mixture, and extruding the material through a microdroplet forming device, producing microdroplets that fall into a slowly stirred hardening bath of an oppositely charged ion or polyelectrolyte solution.

Pre-Processing the Excipient

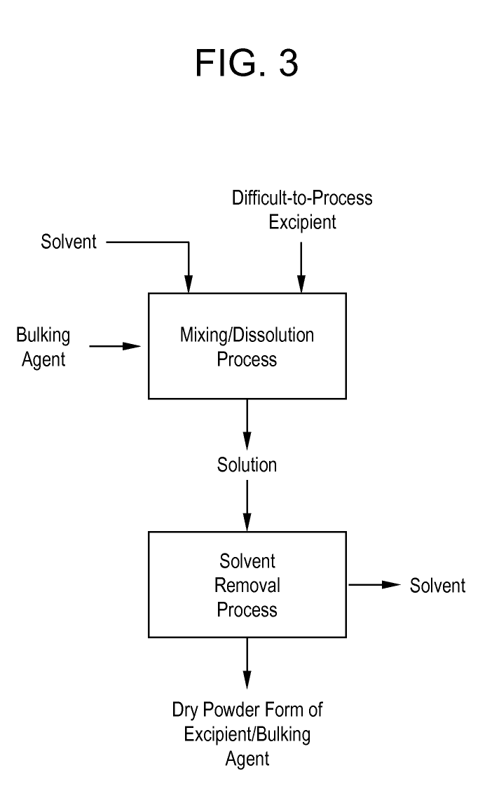

When it is necessary or desirable to convert a liquid, waxy, or otherwise non-friable excipient into a dry powder form suitable for blending and milling, these difficult-to-mill excipient materials are “pre-processed.” In preferred embodiments, the pre-processed excipient that is used or included in the methods and formulations described herein is prepared by (i) dissolving a bulking agent and at least one non-friable excipient in a solvent to form an excipient solution, and then (ii) removing the solvent from the excipient solution to form the pre-processed excipient in dry powder form. See FIG. 3. The dissolution of bulking agent and at least one non-friable excipient in a solvent can be done simply by mixing appropriate amounts of these three components together in any order to form a well mixed solution. A variety of suitable methods of solvent removal known in the art may be used in this process. In one embodiment, the step of removing the solvent comprises spray drying. In another embodiment, the step of removing the solvent comprises lyophilization, vacuum drying, or freeze drying. The pre-processed excipient in dry powder form optionally may be milled prior to blending with the particles comprising pharmaceutical agent.

It is contemplated that the particles of pharmaceutical agent can be blended with one or more pre-processed excipients, and optionally, can be combined with one or more excipients that have not been pre-processed. The particles can be blended with pre-processed excipient(s) either before or after blending with excipient(s) that have not been pre-processed. One or more of the excipients may be jet milled prior to combining with the pharmaceutical agent microparticles.

Blending and Milling

The particles of pharmaceutical agent are blended with one or more other excipient particulate materials, in one or more steps; the resulting blend is then milled; and then the milled blend is blended with another dry powder excipient material. Content uniformity of solid-solid pharmaceutical blends is critical. Comparative studies indicate that the milling of a blend (drug plus excipient) can yield a dry powder pharmaceutical formulation that exhibits an improved dispersibility as compared to a formulation made by milling and then blending or by blending without milling. This improved dispersibility may be realized in a gas stream, as an improved respirable dose from a dry powder inhaler, or in an aqueous liquid environment such as in fluids in the lungs or in a liquid carrier for nebulization. The sequence of the three processing steps is therefore important to the performance of the ultimate pulmonary or nasal dosage form.

1. Blending

The skilled artisan can envision many ways of blending particles in and for the methods and formulations described herein, and the following examples describing how particles may be blended are not intended to limit in any way the methods and formulations described and claimed herein. The blending can be conducted in one or more steps, in a continuous, batch, or semi-batch process. For example, if two or more excipients are used, they can be blended together before, or at the same time as, being blended with the pharmaceutical agent microparticles.

The blending can be carried out using essentially any technique or device suitable for combining the microparticles with one or more other materials (e.g., excipients) effective to achieve uniformity of blend, The blending process may be performed using a variety of blenders. Representative examples of suitable blenders include V-blenders, slant-cone blenders, cube blenders, bin blenders, static continuous blenders, dynamic continuous blenders, orbital screw blenders, planetary blenders, Forberg blenders, horizontal double-arm blenders, horizontal high intensity mixers, vertical high intensity mixers, stirring vane mixers, twin cone mixers drum mixers, and tumble blenders. The blender preferably is of a strict sanitary design required for pharmaceutical products.

Tumble blenders are often preferred for batch operation. In one embodiment, blending is accomplished by aseptically combining two or more components (which can include both dry components and small portions of liquid components) in a suitable container. One example of a tumble blender is the TURBULA™, distributed by Glen Mills Inc., Clifton, N.J., USA, and made by Willy A. Bachofen AG, Maschinenfabrik, Basel, Switzerland.

For continuous or semi-continuous operation, the blender optionally may be provided with a rotary feeder, screw conveyor, or other feeder mechanism for controlled introduction of one or more of the dry powder components into the blender.

2. Milling

The milling step is used to fracture and/or deagglomerate the blended particles, to achieve a desired particle size and size distribution, as well as to insure uniformity of the blend. The skilled artisan can envision many ways of milling particles or blends in the methods and formulations described herein, and the following examples describing how such particles or blend may be milled are not intended to limit in any way the methods and formulations described and claimed herein. A variety of milling processes and equipment known in the art may be used. Examples include hammer mills, ball mills, roller mills, disc grinders and the like. Preferably, a dry milling process is used.

In a preferred technique, the milling comprises jet milling. Jet milling is described for example in U.S. Pat. No. 6,962,006 to Chickering III et al., which is incorporated herein by reference. As used herein, the terms “jet mill” and “jet milling” include and refer to the use of any type of fluid energy impact mills, including spiral jet mills, loop jet mills, and fluidized bed jet mills, with or without internal air classifiers. In one embodiment, the particles are aseptically fed to the jet mill via a feeder, and a suitable gas, preferably dry nitrogen, is used to feed and grind the microparticles through the mill. In another embodiment, the milling process is clean, though not aseptic. Grinding and feed gas pressures can be adjusted based on the material characteristics. Microparticle throughput depends on the size and capacity of the mill. The milled microparticles can be collected by filtration or, more preferably, cyclone.

Processing into Pulmonary or Nasal Dosage Form

The dry powder blend formulations made as described herein are packaged into a pulmonary or nasal dosage form known in the art. The skilled artisan can envision many ways of processing the particle blends in the methods and for the formulations described herein, and the following examples describing how oral dosage forms may be produced are not intended to limit in any way the methods and formulations described and claimed herein. In various embodiments, the blend formulation may be packaged for use in dry powder or liquid suspension form for pulmonary or nasal administration. The formulation can be stored in bulk supply in a dose system for an inhaler or it can be quantified into individual doses stored in unit dose compartments, such as gelatin capsules, blisters, or another unit dose packaging structure known in the art.

The milled blend may optionally undergo additional processes before being finally made into a pulmonary or nasal dosage form. Representative examples of such processes include lyophilization or vacuum drying to further remove residual solvents, temperature conditioning to anneal materials, size classification to recover or remove certain fractions of the particles (i.e., to optimize the size distribution), granulation, and sterilization.

In one embodiment, the dosage form is a dry powder pharmaceutical formulation for pulmonary or nasal administration that includes, or consists substantially of; a blend of a milled blend of (i) microparticles which comprise a pharmaceutical agent, and (ii) excipient particles; and particles of a sugar or sugar alcohol, which particles are larger than the microparticles or excipient particles of the milled blend, wherein the blend which exhibits an increased respirable dose as compared to a respirable dose of combinations of the microparticles, the excipient particles, and the particles of sugar or sugar alcohol which are not blend-of-milled-blend combinations. Examples of the sugar or sugar alcohol include lactose, sucrose, maltose, mannitol, sorbitol, trehalose, galactose, xylitol, erythritol, or a combination thereof In various embodiments, the excipient particles may include a sugar, a sugar alcohol, a starch, an amino acid, or a combination thereof. In one embodiment, the excipient particles and the particles of the sugar or sugar alcohol both comprise lactose. In one embodiment, the pharmaceutical agent has a solubility in water of less than 10 mg/mL at 25° C. In various embodiments, the pharmaceutical agent is budesonide, fluticasone propionate, beclomethasone dipropionate, mometasone, flunisolide, triameinolone acetonide, albuterol, formoterol, salmeterol, cromolyn sodium, ipratropium bromide, testosterone, progesterone, estradiol, or a combination thereof. In a preferred embodiment, the microparticles which comprise pharmaceutical agent have a volume average diameter of less than 10 μm, e.g., less than 5 μm. In one embodiment, the particles of the sugar or sugar alcohol have a volume average diameter between 20 and 500 μm. In various embodiments, the particles of step a) may further comprise a shell material. For example, the shell material may be a biocompatible synthetic polymer.

The Particles and Formulation Components

The pulmonary and nasal dosage formulations made as described herein include mixtures of particles. The mixture generally includes (1) microparticles or nanoparticles that comprise the pharmaceutical agent and that may optionally comprise a shell material, (2) microparticles or nanoparticles of a first excipient material; and (3) particles of a second excipient material, wherein the particles of the second excipient material may or may not be of the same composition as the first excipient material, and wherein the second excipient particles are of a larger size than the microparticles or nanoparticles of the first excipient material.

Particles

The particles comprising pharmaceutical agent that are provided as a starting material in the methods described herein can be provided in a variety of sizes and compositions. As used herein, the term “particles” includes microparticles and nanoparticles, as well as larger particles, e.g., up to 5 mm in the longest dimension. In a preferred embodiment, the particles are microparticles. As used herein, the term “microparticle” encompasses microspheres and microcapsules, as well as microparticles, unless otherwise specified, and denotes particles having a size of 1 to 1000 microns. As used herein, “nanoparticles” have a size of 1 to 1000 nm. In various embodiments, the microparticles or nanoparticles of pharmaceutical agent in the milled pharmaceutical formulation blend have a volume average diameter of less than 100 μm, preferably less than 10 μm, more preferably less than 5 μm. For nasal administration, the particles of pharmaceutical agent in the milled pharmaceutical formulation blend preferably have a number average diameter of between 0.5 μm and 5 mm. For pulmonary administration, the microparticles of pharmaceutical agent in the milled pharmaceutical formulation blend preferably have an aerodynamic diameter of between 1 and 5 μm, with an actual volume average diameter (or an aerodynamic average diameter) of 5 μm or less.

Microparticles may or may not be spherical in shape. Microparticles can be rod like, sphere like, acicular (slender, needle-like particle of similar width and thickness), columnar (long, thin particle with a width and thickness that are greater than those of an acicular particle), flake (thin, flat particle of similar length and width), plate (flat particle of similar length and width but with greater thickness than flakes), lath (long, thin, blade-like particle), equant (particles of similar length, width, and thickness, this includes both cubical and spherical particles), lamellar (stacked plates), or disc like. “Microcapsules” are defined as microparticles having an outer shell surrounding a core of another material, in this case, the pharmaceutical agent. The core can be gas, liquid, gel, solid, or a combination thereof “Microspheres” can be solid spheres, can be porous and include a sponge-like or honeycomb structure formed by pores or voids in a matrix material or shell, or can include multiple discrete voids in a matrix material or shell.

In one embodiment, the particle is formed entirely of the pharmaceutical agent. In another embodiment the particle has a core of pharmaceutical agent encapsulated in a shell. In yet another embodiment, the pharmaceutical agent is interspersed within a shell or matrix. In still another embodiment, the pharmaceutical agent is uniformly mixed within the material comprising the shell or matrix.

The terms “size” or “diameter” in reference to particles refers to the number average particle size, unless otherwise specified. An example of an equation that can be used to describe the number average particle size (and is representative of the method used for the Coulter counter) is shown below:

$$\frac{{\sum }_{i = 1}^{p} {n}_{i} {d}_{i}}{{\sum }_{i = 1}^{p} {n}_{i}}$$

where n=number of particles of a given diameter (d).

As used herein, the term “volume average diameter” refers to the volume weighted diameter average. An example of an equation that can be used to describe the volume average diameter, which is representative of the method used for the Coulter counter is shown below:

$${[ \frac{{\sum }_{i = 1}^{p} {n}_{i} {d}_{i}^{3}}{{\sum }_{i = 1}^{p} {n}_{i}} ]}^{1 / 3}$$

where n=number of particles of a given diameter (d).

Another example of an equation that can be used to describe the volume mean, which is representative of the equation used for laser diffraction particle analysis methods, is shown below:

$$\frac{\sum {d}^{4}}{\sum {d}^{3}}$$

where d represents diameter.

When a Coulter counter method is used, the raw data is directly converted into a number based distribution, which can be mathematically transformed into a volume distribution. When a laser diffraction method is used, the raw data is directly converted into a volume distribution, which can be mathematically transformed into a number distribution.

In the case of a non-spherical particle, the panicles can be analyzed using Coulter counter or laser diffraction methods, with the raw data being converted to a particle size distribution by treating the data as if it came from spherical particles. If microscopy methods are used to assess the particle size for non-spherical particles, the longest axis can be used to represent the diameter (d), with the particle volume (Vₚ) calculated as:

$${V}_{p} = \frac{4 π {r}^{3}}{3}$$

where r is the particle radius (0.5 d), and a number mean and volume mean are calculated using the same equations used for a Coulter counter.

As used herein, the term “aerodynamic diameter” refers to the equivalent diameter of a sphere with density of 1 g/mL were it to fall under gravity with the same velocity as the particle analyzed. The values of the aerodynamic average diameter for the distribution of particles are reported. Aerodynamic diameters can be determined on the dry powder using an Aerosizer (TSI), which is a time of flight technique, or by cascade impaction, or liquid impinger techniques. Where an Andersen cascade impaction performed at 60 lpm is described, the respirable dose is the amount of drug that has passed through Stage-0 (the cumulative amount of drug on Stages 1 through the filter).

Particle size analysis can be performed on a Coulter counter, by light microscopy, scanning electron microscopy, transmission electron microscopy, laser diffraction methods, light scattering methods or time of flight methods. Where a Coulter counter method is described, the powder is dispersed in an electrolyte, and the resulting suspension analyzed using a Coulter Multisizer II fitted with a 50 -μm aperture tube. Where a laser diffraction method is used, the powder is dispersed in an aqueous medium and analyzed using a Coulter LS230, with refractive index values appropriately chosen for the material being tested.

Aerodynamic particle size analysis can be performed using a cascade impactor, a liquid impinger or time of flight methods.

As used herein, the term “respirable dose” refers to a dose of drug that has an aerodynamic size such that particles or droplets comprising the drug are in the aerodynamic size range that would be expected to reach the lung upon inhalation. Respirable dose can be measured using a cascade impactor, a liquid impinger, or time of flight methods.

1. Pharmaceutical Agent

The pharmaceutical agent is a therapeutic, diagnostic, or prophylactic agent. It may be an active pharmaceutical ingredient (API) and may be referred to herein generally as a “drug” or “active agent.” The pharmaceutical agent may be present in an amorphous state, a crystalline state, or a mixture thereof. The pharmaceutical agent may be labeled with a detectable label such as a fluorescent label, radioactive label or an enzymatic or chromatographically detectable agent.

The methods can be applied to a wide variety of therapeutic, diagnostic and prophylactic agents that may be suitable for pulmonary or nasal administration. For example, the pharmaceutical agent can be a bronchodilator, a steroid, an antibiotic, an antiasthmatic, an antineoplastic, a peptide, or a protein. In one embodiment, the pharmaceutical agent comprises a corticosteroid, such as budesonide, fluticasone propionate, beclomethasone dipropionate, mometasone, flunisolide, or triamcinolone acetonide. In another embodiment, the pharmaceutical agent comprises albuterol, formoterol, salmeterol, cromolyn sodium, ipratropium bromide, testosterone, progesterone, estradiol, or a combination thereof.

Representative examples of suitable drugs include the following categories and examples of drugs and alternative forms of these drugs such as alternative salt forms, free acid forms, free base forms, and hydrates:
analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, and meprobamate);
antiasthmatics;
antibiotics (e.g., neomycin, streptomycin, chloramphenicol, cephalosporin, ampicillin, penicillin, tetracycline, and ciprofloxacin);
antidepressants (e.g., nefopam, oxypertine, doxepin, amoxapine, trazodone, amitriptyline, maprotiline, phenelzine, desipramine, nortriptyline, tranylcypromine, fluoxetine, imipramine, imipramine pamoate, isocarboxazid, trimipramine, and protriptyline);
antidiabetics (e.g., biguanides and sulfonylurea derivatives);
antifungal agents (e.g., griseofulvin, ketoconazole, itraconizole, virconazole, amphotericin B, nystatin, and candicidin);
antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);
anti-inflammatories (e.g., (non-steroidal) celecoxib, rofecoxib, indomethacin, ketoprofen, flurbiprofen, naproxen, ibuprofen, ramifenazone, piroxicam, (steroidal) cortisone, dexamethasone, fluazacort, hydrocortisone, prednisolone, and prednisone);
antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, antiapoptotic agents, etoposide, camptothecin and derivatives thereof, phenesterine, paclitaxel and derivatives thereof, docetaxel and derivatives thereof; vinblastine, vincristine, tamoxifen, and piposulfan);
antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);
immunosuppressive agents (e.g., cyclosporine, azathioprine, mizoribine, and FK506 (tacrolimus), sirolimus);
antimigraine agents (e.g., ergotamine, propanolol, and dichloralphenazone);
sedatives/hypnotics (e.g., barbiturates such as pentobarbital, pentobarbital, and secobarbital; and benzodiazapines such as flurazepam hydrochloride, and triazolam);
antianginal agents (e.g., beta-adrenergic blockers; calcium channel blockers such as nifedipine, and diltiazem; and nitrates such as nitroglycerin, and erythrityl tetranitrate);
antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, lithium citrate, prochlorperazine, aripiprazole, and risperdione);
antimanic agents (e.g., lithium carbonate);
antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, flecainide acetate, tocainide, and lidocaine);
antiathritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium);
antigout agents (e.g., colchicine, and allopurinol);
anticoagulants (e.g., desirudin, heparin, low molecular weight heparin, heparin sodium, and warfarin sodium);
thrombolytic agents (e.g., urokinase, streptokinase, and alteplase);
anitfibrinolytic agents (e.g., aminocaproic acid);
hemorheologic agents (e.g., pentoxifylline);
antiplatelet agents (e.g., aspirin, clopidogrel);
anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, paramethadione, ethotoin, phenacemide, secobarbitol sodium, clorazepate dipotassium, oxcarbazepine and trimethadione);
antiparkinson agents (e.g., ethosuximide);
anthistamines/antipruritics (e.g., hydroxyzine, diphenhydramine, chlorpheniramine, brompheniramine maleate, cyproheptadine hydrochloride, terfenadine, clemastine fumarate, azatadine, tripelennamine, dexchlorpheniramine maleate, methdilazine);
agents useful for calcium regulation (e.g., calcitonin, and parathyroid hormone);
antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, clarithromycin and colistin sulfate);
antiviral agents (e.g., interferons, zidovudine, amantadine hydrochloride, ribavirin, and acyclovir);
antimicrobials (e.g., cephalosporins such as ceftazidime; penicillins; erythromycins; and tetracyclines such as tetracycline hydrochloride, doxycycline hyclate, and minocycline hydrochloride, azithromycin, clarithromycin);
anti-infectives (e.g., GM-CSF);
bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; salbutamol; ipratropium bromide: ketotifen; salmeterol; xinafoate; terbutaline sulfate; theophylline; nedocromil sodium; metaproterenol sulfate; albuterol);
inhalant corticosteroids (e.g., beclomethasone dipropionate (BDP), beclomethasone dipropionate monohydrate; budesonide, triamcinolone; flunisolide; fluticasone proprionate; mometasone);
steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);
hypoglycemic agents (e.g., human insulin, purified beef insulin, purified pork insulin, glyburide, chlorpropamide, glipizide, tolbutamide, and tolazamide);
hypolipidemic agents (e.g., clofibrate, dextrothyroxine sodium, probucol, pravastitin, atorvastatin, lovastatin, and niacin);
proteins (e.g., DNase, alginase, superoxide dismutase, and lipase);
nucleic acids (e.g., sense or anti-sense nucleic acids encoding any therapeutically useful protein, including any of the proteins described herein);
agents useful for erythropoiesis stimulation (e.g., erythropoietin); antiulcer/antireflux agents (e.g., famotidine, cimetidine, and ranitidine hydrochloride);
antinauseants/antiemetics (e.g., meclizine hydrochloride, nabilone, prochlorperazine, dimenhydrinate, promethazine hydrochloride, thiethylperazine, and scopolamine);
oil-soluble vitamins (e.g., vitamins A, D, E, K, and the like); as well as other drugs such as mitotane, halonitrosoureas, anthrocyclines, and ellipticine. A description of these and other classes of useful drugs and a listing of species within each class can be found in Martindale, The Extra Pharmacopoeia, 30th Ed. (The Pharmaceutical Press, London 1993).

In particular examples of the methods and formulations described herein, the drug is selected from among enoxaprin, ondansetron, sumatriptan, sildenofil, albuterol, dornase alpha, iloprost, heparin, low molecular weight heparin, and desirudin.

In one embodiment, the pharmaceutical agent used in the methods and formulations described herein is a hydrophobic compound, particularly a hydrophobic therapeutic agent. Examples of such hydrophobic drugs include celecoxib, rofecoxib, paclitaxel, docetaxel, acyclovir, alprazolam, amiodaron, amoxicillin, anagrelide, bactrim, biaxin, budesonide, bulsulfan, carhamazepine, ceftazidime, cefprozil, ciprofloxicin, clarithromycin, clozapine, cyclosporine, diazepam, estradiol, etodolac, famciclovir, fenofibrate, fexofenadine, gemcitabine, ganciclovir, itraconazole, lamotrigine, loratidine, lorazepam, meloxicam, mesalamine, minocycline, modafinil, nabumetone, nelfinavir mesylate, olanzapine, oxcarbazepine, phenytoin, propofol, ritinavir, SN-38, sulfamethoxazol, sulfasalazine, tracrolimus, tiagabine, tizanidine, trimethoprim, valium, valsartan, voriconazole, zafirlukast, zileuton, and ziprasidone.

Additional examples of drugs that may be useful in the methods and formulations described herein include ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered water-soluble.

Other examples of possible drugs include adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amiodipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D₃ and related analogues, finasteride, quetiapine fumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfioxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinotone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam.

In another embodiment, the pharmaceutical agent may be a contrast agent for diagnostic imaging. For example, the diagnostic agent may be an imaging agent useful in positron emission tomography (PET), computer assisted tomography (CAT), single photon emission computerized tomography, x-ray, fluoroscopy, magnetic resonance imaging (MRI), or ultrasound imaging. Microparticles loaded with these agents can be detected using standard techniques available in the art and commercially available equipment. Examples of suitable materials for use as MRI contrast agents include soluble iron compounds (ferrous gluconate, ferric ammonium citrate) and gadolinium-diethylenetriaminepentaacetate (Gd-DTPA).

2. Shell Material

The particles that include the pharmaceutical agent may also include a shell material. The shell material can be water soluble or water insoluble, degradable, erodible or non-erodible, natural or synthetic, depending for example on the particular dosage form selected and release kinetics desired. Representative examples of types of shell materials include polymers, amino acids, sugars, proteins, carbohydrates, and lipids. Polymeric shell materials can be erodible or non-erodible, natural or synthetic. In general, synthetic polymers may be preferred due to more reproducible synthesis and degradation. Natural polymers also may be used. A polymer may be selected based on a variety of performance factors, including shelf life, the time required for stable distribution to the site where delivery is desired, degradation rate, mechanical properties, and glass transition temperature of the polymer.

Representative examples of synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polyalkylenes such as polyethylene and polypropylene, polyalkylene glycols such as poly(ethylene glycol), polyalkylene oxides such as poly(ethylene oxide), polyvinylpyrrolidone, poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, poly(butyric acid), poly(valeric acid), poly(lactide-co-caprolactone), blends and copolymers thereof.

Examples of preferred natural polymers include proteins such as albumin. The in vivo stability of the matrix can be adjusted during the production by using polymers such as polylactide-co-glycolide copolymerized with polyethylene glycol (PEG). PEG, if exposed on the external surface, may extend the time before these materials are phagocytosed by the reticuloendothelial system (RES), as it is hydrophilic and has been demonstrated to mask RES recognition.

Representative amino acids that can be used in the shell include both naturally occurring and non-naturally occurring amino acids. The amino acids can be hydrophobic or hydrophilic and may be D amino acids, L amino acids or racemic mixtures. Amino acids that can be used include glycine, arginine, histidine, threonine, asparagine, aspartic acid, serine, glutamate, proline, cysteine, methionine, valine, leucine, isoleucine, tryptophan, phenylalanine, tyrosine, lysine, alanine, and glutamine. The amino acid can be used as a bulking agent, or as an anti-crystallization agent for drugs in the amorphous state, or as a crystal growth inhibitor for drugs in the crystalline state or as a wetting agent. Hydrophobic amino acids such as leucine, isoleucine, alanine, glycine, valine, proline, cysteine, methionine, phenylalanine, or tryptophan are more likely to be effective as anticrystallization agents or crystal growth inhibitors. In addition, amino acids can serve to make the shell have a pH dependency that can be used to influence the pharmaceutical properties of the shell such as solubility, rate of dissolution or wetting.

The shell material can be the same as or different from the excipient material.

Excipients Bulking Agents

The drug particles are blended with one or more excipients particles. The term “excipient” refers to any non-active pharmaceutically acceptable ingredient of the formulation intended to facilitate handling, stability, wettability, release kinetics, and/or pulmonary or nasal administration of the pharmaceutical agent. The excipient may be a pharmaceutically acceptable carrier or bulking agent as known in the art. The excipient may comprise a shell material, protein, amino acid, sugar or other carbohydrate, starch, lipid, or combination thereof. In one embodiment, the excipient is in the form of microparticles. In one embodiment, the excipient microparticles have a volume average size between about 5 and 500 μm.

In one embodiment, the excipient is a pre-processed excipient. A pre-processed excipient is one that initially cannot be readily handled in a dry powder form and that has been converted into a form suitable for dry powder processing (e.g., for milling or blending). A preferred pre-processing process is described above. In preferred embodiments, the excipient of the pre-processed excipient comprises a liquid, waxy, non-crystalline compound, or other non-friable compound. In a preferred embodiment, the non-friable excipient comprises a surfactant, such as a waxy or liquid surfactant. By “liquid,” it is meant that the material is a liquid at ambient temperature and pressure conditions (e.g., 15-25° C. and atmospheric pressure). Examples of such surfactants include docusate sodium (DSS), polysorbates, phospholipids, and fatty acids. In a preferred embodiment, the surfactant is a Tween or other hydrophilic non-ionic surfactant. The pre-processed excipient further includes at least one bulking agent. In preferred embodiments, the bulking agent comprises at least one sugar, sugar alcohol, starch, amino acid, or combination thereof. Examples of suitable bulking agents include lactose, sucrose, maltose, mannitol, sorbitol, trehalose, galactose, xylitol, erythritol, and combinations thereof In one particular embodiment of the methods described herein, a saccharide (e.g., mannitol) and a surfactant (e.g., TWEEN™ 80) are blended in the presence of water and the water is then removed by spray-drying or lyophilization, yielding a pre-processed excipient of a saccharide and a surfactant. The pre-processed saccharide/surfactant blend is then blended with microparticles formed of or including a pharmaceutical agent. In one case, the saccharide is provided at between 100 and 200% w/w microparticles, while the surfactant is provided at between 0.1 and 10% w/w microparticles. In one case, the saccharide is provided with a volume average particle size between 10 and 500 μm.

Representative amino acids that can be used as excipients include both naturally occurring and non-naturally occurring amino acids. The amino acids can be hydrophobic or hydrophilic and may be D amino acids, L amino acids or racemic mixtures. Amino acids which can be used include glycine, arginine, histidine, threonine, asparagine, aspartic acid, serine, glutamate, proline, cysteine, methionine, valine, leucine, isoleucine, tryptophan, phenylalanine, tyrosine, lysine, alanine, and glutamine. The amino acid can be used as a bulking agent, as a wetting agent, or as a crystal growth inhibitor for drugs in the crystalline state. Hydrophobic amino acids such as leucine, isoleucine, alanine, glycine, valine, proline, cysteine, methionine, phenylalanine, tryptophan are more likely to be effective as crystal growth inhibitors. In addition, amino acids can serve to make the matrix have a pH dependency that can be used to influence the pharmaceutical properties of the matrix, such as solubility, rate of dissolution, or wetting.

Examples of excipients include surface active agents and osmotic agents known in the art. Examples include sodium desoxycholate; sodium dodecylsulfate; polyoxyethylene sorbitan fatty acid esters, e.g., polyoxyethylene 20 sorbitan monolaurate (TWEEN™ 20), polyoxyethylene 4 sorbitan monolaurate (TWEEN™ 21), polyoxyethylene 20 sorbitan monopalmitate (TWEEN™ 40), polyoxyethylene 20 sorbitan monooleate (TWEEN™ 80); polyoxyethylene alkyl ethers, e.g., polyoxyethylene 4 lauryl ether (BRIJ™ 30), polyoxyethylene 23 lauryl ether (BRIJ 35), polyoxyethylene 10 oleyl ether (BRIJ™ 97); polyoxyethylene glycol esters, e.g., poloxyethylene 8 stearate (MYRJ™ 45), poloxyethylene 40 stearate (MYRJ™ 52); Tyloxapol; Spans (e.g., SPAN80, SPAN85); phospholipids, fatty acids, and mixtures thereof.

The invention can further be understood with reference to the following non-limiting examples.

### EXAMPLES

A TURBULA™ inversion mixer (model: T2F) was used for blending. A Fluid Energy Aljet jet mill was used. The mill used dry nitrogen gas as the injector and grinding gases. In the studies, the dry powder was fed manually into the jet mill, and hence the powder feed rate was not constant. Although the powder feeding was manual, the feed rate was calculated to be approximately 1 to 5 g/min. for all Examples. Feed rate is the ratio of total material processed in one batch to the total batch time.

The following materials were used in the examples: mannitol (Spectrum Chemicals, New Brunswick, N.J., unless otherwise indicated), TWEEN™ 80 (Spectrum Chemicals, New Brunswick, N.J.), celecoxib (Onbio, Ontario, Canada), Plasdone-C 15 (International Specialty Products, Wayne, N.Y.), budesonide (Byron Chemical Company, Long Island, N.Y.), dipalmitoyl phosphatidylcholine (DPPC) (Chemi S.p.a., Milan, Italy, unless otherwise indicated), PLGA (Boehringer Ingelheim Fine Chemicals, Ingelheim, Germany), ammonium bicarbonate (Spectrum Chemicals, Gardenia, Calif.), methylene chloride (EM Science, Gibbstown, N.J.), Fluticasone propionate (Cipla Ltd., Mumbai, India), and lactose (Pharmatose 325M, DMV International, The Netherlands). The TWEEN™ 80 is hereinafter referred to as “Tween80.” The volume average diameter of lactose (Pharmatose 325M) was determined to be approximately 68 μm by dry powder particle sizing using a Malvern Mastersizer (Malvern Instruments Ltd., United Kingdom).

An Andersen cascade impactor (ACI), equipped with a pre-separator, was used to determine the aerodynamic particle size distribution of microparticles, either alone or blended with lactose, as emitted from a dry powder inhaler. The plates for each stage of the ACI, as well as the pre-separator, were pre-coated with propylene glycol. A flow rate of 60 L/min was used. Five “puffs” from the inhaler were collected in the ACI for each experiment. For such analysis, a single puff consisted of a gelatin capsule filled with the powder being tested. (For example, with 824 μg budesonide per puff or 500 μg of fluticasone propionate per puff.) After the five puffs, the impactor was disassembled, and the components were rinsed or soaked with a solvent (50% ethanol in water for budesonide studies, 65% acetonitrile in water for fluticasone). The resulting material was filtered, and analyzed for drug content by HPLC. Quantitation was performed using an 8 point calibration curve (e.g., over the range of 0.15 to 70 μg/mL for budesonide and 0.12 to 33.60 μg/mL for fluticasone propionate). The “Respirable Dose” was the quantity of material from Stage 1 through the filter, The HPLC conditions used for budesonide analysis were a J'sphere column (CDS-H80 250×4.6 mm) with ethanol:water (64:36) as an eluant, a flow rate of 0.8 mL/min, a column temperature of 42° C., a sample temperature of 4° C., an injection volume of 100 μl, and a detector wavelength of 254 nm. The HPLC conditions used for fluticasone propionate analysis were a J'sphere column (ODS-H80 250×4.6 mm) with acetonitrile:water (68:32) as an eluant, a flow rate of 1 mL/min, a column temperature of 42° C., a sample temperature of 4° C., an injection volume of 100 μl, and a detector wavelength of 238 nm.

### Example 1

### Microparticle Dispersibility Comparison of Reconstituted Celecoxib Blend Formulations Made by Different Methods

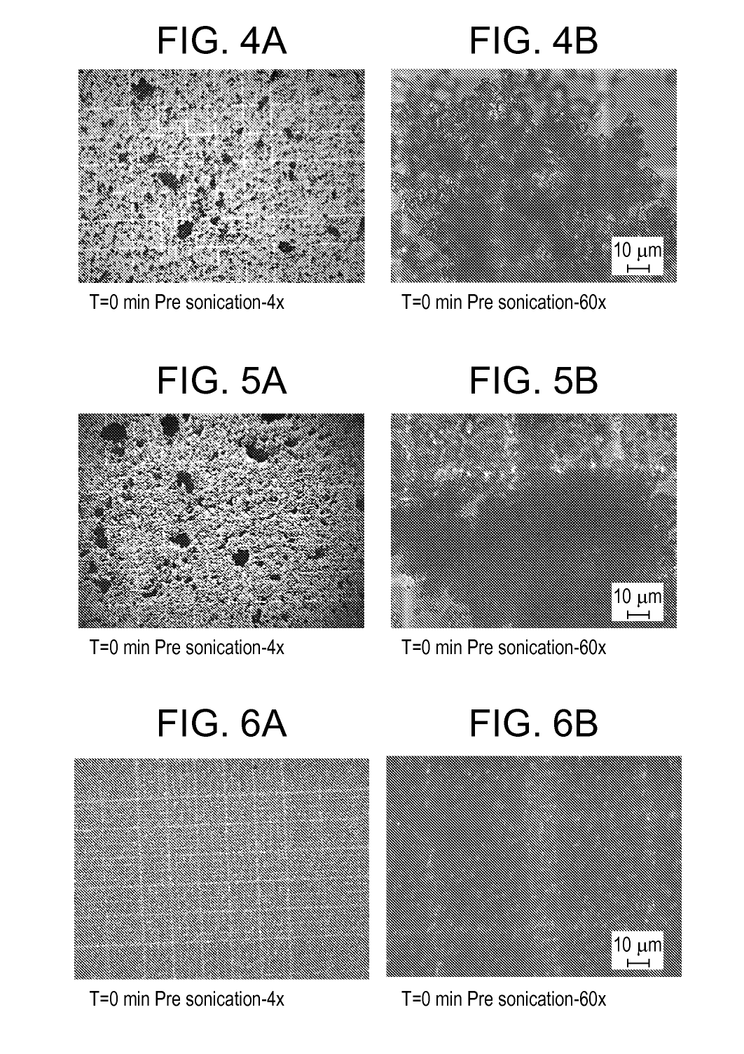

A dry powder blend formulation was prepared by one of three different processes and then reconstituted in water. The dry powder blend consisted of celecoxib, mannitol, Plasdone-C15, and Tween80 at a ratio of 5:10:1:1. The mannitol (Pearlitol 100SD from Roquette America Inc., Keokuk, Iowa) and the Tween80 were pre-processed, at a ratio of 10:1, by dissolution in water (18 g mannitol and 1.8 g Tween80 in 104 mL water) followed by freezing at −80° C. and lyophilization. The three processes compared were (1) blending the celecoxib and pre-processed excipient particles without milling, (2) separately milling the celecoxib particles and then blending the milled particles with pre-processed excipients, or (3) blending the celecoxib and pre-processed excipient particles and then milling the resulting blend. The resulting blends were reconstituted in water using shaking, and analyzed by light scattering using an LS230 Laser Diffraction Particle Size Analyzer (Beckman Coulter, Fullerton, Calif.). The particles' sizes from each of the three processes were compared. The size results are shown in Table 1, along with visual evaluations of the quality of the suspensions. FIGS. 4A-B show the microscopy results of reconstituted celecoxib from a blend of excipient particles and celecoxib particles (Process 1). FIGS. 5A-B show the microscopy results of reconstituted celecoxib from a blend of excipient particles and milled celecoxib panicles (Process 2). FIGS. 6A-B show the microscopy results of reconstituted celecoxib from a jet milled blend of excipient particles and celecoxib particles (Process 3).

TABLE 1
Results of Particle Size Analysis and Observations Following Reconstitution

| | LS230 Particle Size | | | |
|---|---|---|---|---|
| | Analysis T = 0 | | Visual Evaluation | |
| | Post Reconstitution | | of Suspension | |
| | Volume | % <90 | Post Reconstitution | |
| Sample | mean (μm) | (μm) | T = 0 | T = 60 min |
| Celecoxib | 56.27 | 156.95 | Fine suspension with | Fine suspension with |
| Particles Blended | | | many small | many small |
| | | | macroparticles | macroparticles |
| Blend of Jet | 58.98 | 153.08 | Fine suspension with | Fine suspension with |
| Milled Celecoxib | | | many small | many small |
| Particles | | | macroparticles | macroparticles |
| Jet Milled Blend | 5.45 | 9.12 | Fine suspension with | Fine Suspension |
| of Celecoxib | | | very few small | |
| Particles | | | macroparticles | |

These results strongly indicate that the processing method impacts the resulting suspension quality. The results also indicate the advantages offered by milled blend formulations as compared to the formulations made by the other methods.

Jet milling of blended celecoxib particles led to a powder which was better dispersed, as indicated by the resulting fine suspension with a few macroscopic particles. This suspension was better than the suspensions of the unprocessed celecoxib microparticles and the blended celecoxib microparticles.

The light microscope images (FIGS. 4-6) of the suspensions indicate no significant change to individual particle morphology just to the ability of the individual particles to disperse as indicated by the more uniform size and increased number of suspended microparticies following both blending and jet milling as compared to the two other microparticle samples.

### Example 2

### Production of Microparticles Containing Budesonide

Two different samples of budesonide were prepared. Sample 2a was prepared as follows: 8.0 g of PLGA, 0.48 g of DPPC, and 2.2 g of budesonide were dissolved in 392 mL of methylene chloride, and 1.1 g of ammonium bicarbonate was dissolved in 10.4 g of water. The ammonium bicarbonate solution was combined with the budesonide/PLGA solution and emulsified using a rotor-stator homogenizer. The resulting emulsion was spray dried on a benchtop spray dryer using an air-atomizing nozzle and nitrogen as the drying gas. Spray drying conditions were as follows: 20 mL/min emulsion flow rate, 60 kg/hr drying gas rate and 21° C. outlet temperature. The product collection container was detached from the spray dryer and attached to a vacuum pump, where the collected product was dried for 53 hours.

Sample 2b was prepared as follows: 36.0 g of PLGA, 2.2 g of DPPC, and 9.9 g of budesonide were dissolved in 1764 mL of methylene chloride, and 3.85 g of ammonium bicarbonate was dissolved in 34.6 g of water. The ammonium bicarbonate solution was combined with the budesonide/PLGA solution and emulsified using a rotor-stator homogenizer. The resulting emulsion was spray dried on a benchtop spray dryer using an air-atomizing nozzle and nitrogen as the drying gas. Spray drying conditions were as follows: 20 mL/min emulsion flow rate, 60 kg/hr drying gas rate and 21° C. outlet temperature. The product collection container was detached from the spray dryer and attached to a vacuum pump, where the collected product was dried for 72 hours.

### Example 3

### Production of Microparticles Comprising Fluticasone Propionate

Microparticles containing fluticasone propionate were made as follows: 3.0 g of PLGA, 0.36 g of DPPC, and 2.2 g of fluticasone propionate were dissolved in 189 mL of methylene chloride, and 0.825 g of ammonium bicarbonate was dissolved in 7.6 g of water. The ammonium bicarbonate solution was combined with the fluticasone priopionate/PLGA solution and emulsified using a rotor-stator homogenizer. The resulting emulsion was spray dried on a benchtop spray dryer using an air-atomizing nozzle and nitrogen as the drying gas. Spray drying conditions were as follows: 20 mL/min emulsion flow rate, 60 kg/hr drying gas rate and 20° C. outlet temperature. The product collection container was detached from the spray dryer and attached to a vacuum pump, where the collected product was dried for 49 hours. Two batches made according to the above method were manually blended to create a single combined batch.

### Example 4

### Effect of Blending and Milling on Aerodynamic Particle Size Distribution and Storage Stability for Microparticles Comprising Budesonide

Three different samples of budesonide formulations were prepared. Sample 4a was prepared as follows to make a blend of microparticles (the “Blend”): Microparticles as made in Sample 2a (5.25 g) and 27.6 g of lactose (Pharmatose 325M) were blended on a Turbula blender for 30 minutes at 96 rpm.

Sample 4b was prepared as follows to make ajet milled blend of microparticles (the Jet Milled Blend, “JMB”): Microparticles as made in Sample 2b (6.00 g) and 31.52 g of lactose (Pharmatose 325M) were blended on a Turbula blender for 30 minutes at 96 rpm. The resulting dry blended powder was then was fed manually into a Hosokawa spiral jet mill (injector gas pressure 3 bar, grinding gas pressure 2 bar).

Sample 4c was prepared as follows to make a blend of a jet milled blend of microparticles (the Blend of Jet Milled Blend, “BJMB”): Microparticles as made in Sample 2a (6.01 g) and 15.05 g of lactose (Pharmatose 325M) were blended on a Turbula blender for 30 minutes at 96 rpm. The resulting dry blended powder was then was fed manually into a Hosokawa spiral jet mill (injector gas pressure 3 bar, grinding gas pressure 2 bar). Then, the resulting milled blend (16.39 g) and 12.88 g of lactose (Pharmatose 325M) were blended in a 725 mL vessel on a Turbula blender for 30 minutes at 96 rpm.

Samples 4a-c were stored at 30° C. and 60% RH in open containers. At select time-points, the materials were filled into gelatin capsules (824 μg nominal budesonide per capsule) and analyzed by Andersen cascade impaction using a Cyclohaler dry powder inhaler. The results are shown in Table 2.

TABLE 2
Respirable Dose of Dry Powder Formulation Made by Different Methods

| | | | Respirable | % Change in |
|---|---|---|---|---|
| | | Respirable Dose | Dose | Respirable |
| | | (μg/puff) | (μg/puff) | Dose over 3 |
| Material | Process | T = 0 | T = 3 months | Months |
| Example 4a | Blend | 204.7 | 60.95 | −70% |
| Example 4b | JMB | 182.6 | 168.1 | −8% |
| Example 4c | BJMB | 261.0 | 163.9 | −20% |

The data in Table 2 show that the highest respirable dose at T=0 is seen for Sample 4c (a BJMB material). The data in Table 2 also shows that the smallest change in respirable dose after 3 months of storage at 30° C./60% RH is seen for Sample 4b (a JMB material). Thus, if materials are sensitive to heat or humidity, the use of a material that is a milled blend of (i) microparticles comprising a pharmaceutical agent and (ii) excipient particles (e.g., 325 M lactose) may be preferred.

### Example 5

### Effect of Blending and Milling on Aerodynamic Particle Size Distribution and Stability for Microparticles Comprising Fluticasone Propionate

Three different samples of fluticasone propionate formulations were prepared. Sample 5a was prepared as follows to make a blend of microparticles: Microparticles from Example 3 (0.51 g) and 4.49 g of lactose (Pharmatose 325M) were blended on a Turbula blender for 60 minutes at 96 rpm.

Sample 5b was prepared as follows to make ajet milled blend of microparticles: Microparticles from Example 3 (0.765 g) and 6.735 g of lactose (Pharmatose 325M) were blended on a Turbula blender for 60 minutes at 96 rpm. The resulting dry blended powder was then fed manually into a Fluid Energy Aljet spiral jet mill (injector gas pressure 8 bar, grinding gas pressure 4 bar).

Sample 5c was prepared as follows to make a blend of a jet milled blend of microparticles: Microparticles from Example 4 (1.82 g) and 3.18 g of lactose (Pharmatose 325M) were blended on a Turbula blender for 30 minutes at 96 rpm. The resulting dry blended powder was then was fed manually into a Fluid Energy Aljet spiral jet mill (injector gas pressure 8 bar, grinding gas pressure 4 bar). Then, the resulting milled blend (2.50 g) and 6.50 g of lactose (Pharmatose 325M) were blended on a Turbula blender for 30 minutes at 96 rpm.

Material from Samples 5a-c were filled into gelatin capsules (500 μg fluticasone propionate nominal per capsule), and then stored at 30° C. and 60% RH in closed containers. At select time-points, the materials were analyzed by Andersen cascade impaction using a Cyclohaler dry powder inhaler. The results are shown in Table 3.

TABLE 3
Respirable Dose of Dry Powder Formulation Made by Different Methods

| | | Respirable | | |
|---|---|---|---|---|
| | | Dose | Respirable Dose | % Change in |
| | | (μg/puff) | (μg/puff) | Respirable Dose |
| Material | Process | T = 0 | T = 3 months | over 3 months |
| Sample 5a | Blend | 189.9 | 70.4 | −63% |
| Sample 5b | JMB | 184.9 | 152.7 | −17% |
| Sample 5c | BJMB | 219.3 | 93.9 | −57% |

The data in Table 3 shows that the highest respirable dose at T=0 is seen for Sample 5c, which is a blend of a jet milled blend. The data in Table 3 also show that the smallest change in respirable dose after 3 months of storage at 30° C./60% RH: is seen for Sample 5b, which is a jet milled blend. Thus, if a dry powder formulation is sensitive to heat or humidity, the use of a material that is a milled blend of (i) microparticles comprising a pharmaceutical agent and (ii) excipient particles (e.g., Pharmatose 325M lactose) is preferred.

### Example 6

### Effect on Respirable Dose of Adding DPPC to a Blend to Make a Jet Milled Blend of Microparticles Comprising Fluticasone Propionate

Two different samples of fluticasone propionate formulations were prepared. Sample 6a was prepared as follows to make a jet milled blend of microparticles of fluticasone propionate in the absence of DPPC (the “JMB without DPPC”): Fluticasone propionate (20.83 mg) and 980.68 mg of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar respectively).

Sample 6b was prepared as follows to make a jet milled blend of microparticles of fluticasone propionate with DPPC added to the blend (the “JMB with DPPC”): Fluticasone propionate (20.13 mg), DPPC (20.88 mg) and 960.60 mg of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar).

The materials were filled into gelatin capsules (500 μg nominal fluticasone propionate per capsule) and analyzed by Andersen cascade impaction using a Cyclohaler dry powder inhaler. The results are shown in Table 4.

TABLE 4
Respirable Dose of Dry Powder Formulations

| | | Respirable | Respirable Dose | Change in |
|---|---|---|---|---|
| | | Dose | as a Percent of | Respired Dose |
| Material | Formulation | (μg/puff) | Nominal Dose | Due to DPPC |
| Example 6a - Rep 1 | JMB without DPPC | 110.6 | 22.12 | |
| Example 6a - Rep 2 | JMB without DPPC | 116.4 | 23.28 | |
| Example 6a - Rep 3 | JMB without DPPC | 99.9 | 19.98 | |
| Example 6a - Avg. | JMB without DPPC | 109.0 | 21.80 | |
| Example 6b - Rep 1 | JMB without DPPC | 153.4 | 30.68 | |
| Example 6b - Rep 2 | JMB without DPPC | 178.5 | 35.70 | |
| Example 6b - Rep 3 | JMB without DPPC | 162.9 | 32.58 | |
| Example 6b - Avg. | JMB without DPPC | 164.9 | 32.98 | +51% |

The data in Table 4 show that the highest respirable dose is seen for Sample 6b, where DPPC is added to the blend prior to milling.

### Example 7

### Effect on Respirable Dose of Adding DPPC to a Blend to Make a Jet Milled Blend of Microparticles Comprising Budesonide

Two different samples of budesonide formulations were prepared. Sample 7a was prepared as follows to make a jet milled blend of microparticles of budesonide in the absence of DPPC (the “JMB without DPPC”): Budesonide (0.165 g) and 4.835 g of lactose were blended on a Turbula blender for 10 minutes at 96 rpm. The resulting dry blended powder was then was fed manually into a Fluid Energy Aljet spiral jet mill (injector gas pressure 8 bar, grinding gas pressure 4 bar).

Sample 7b was prepared as follows to make a jet milled blend of microparticles of budesonide with DPPC added to the blend (the “JMB with DPPC”): Budesonide (0.165 g), DPPC (0.165 g) and 4.67 g of lactose were blended on a Turbula blender for 10 minutes at 96 rpm. The resulting dry blended powder was then was fed manually into a Fluid Energy Aljet spiral jet mill (injector gas pressure 8 bar, grinding gas pressure 4 bar).

The materials were filled into gelatin capsules (825 μg nominal budesonide per capsule) and analyzed by Andersen cascade impaction using a Cyclohaler dry powder inhaler. The results are shown in Table 5.

TABLE 5
Respirable Dose of Dry Powder Formulations

| | | | Respirable Dose | Change in |
|---|---|---|---|---|
| | | Respirable | as a Percent of | Respired Dose |
| Material | Formulation | Dose (μg/puff) | Nominal Dose | Due to DPPC |
| Example 7a - Rep 1 | JMB without DPPC | 205.2 | 24.87 | |
| Example 7a - Rep 2 | JMB without DPPC | 241.8 | 29.31 | |
| Example 7a - Avg | JMB without DPPC | 223.5 | 27.09 | |
| Example 7b - Rep 1 | JMB with DPPC | 349.4 | 42.35 | |
| Example 7b - Rep 2 | JMB with DPPC | 404.5 | 49.03 | |
| Example 7b - Avg | JMB with DPPC | 377.0 | 45.70 | +69% |

The data in Table 5 show that the highest respirable dose is seen for Sample 7b, where DPPC is added to the blend prior to milling.

### Example 8

### Effect on Respirable Dose of Adding DPPC to a Blend to Make a Blend of Jet Milled Blend of Microparticles Including Fluticasone Priopionate

Two different samples of fluticasone propionate formulations were prepared. Sample 8a was prepared as follows to make a blend of a jet milled blend of microparticles of fluticasone propionate in the absence of DPPC (the “BJMB without DPPC”): Fluticasone propionate (40.94 mg) and 960.35 mg of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar respectively). Then, the resulting milled blend (780 mg) and 782.40 mg of lactose were blended on a Turbula blender for 10 minutes at 96 rpm.

Sample 8b was prepared as follows to make a blend of a jet milled blend of microparticles of fluticasone propionate with DPPC added to the blend prior to milling (the “BJMB with DPPC”): Fluticasone propionate (38.44 mg), DPPC (37.58 mg) and 923.79 mg of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar). Then, the resulting milled blend (750 mg) and 692.23 mg of lactose were blended on a Turbula blender for 10 minutes at 96 rpm.

The materials were filled into gelatin capsules (500 μg nominal fluticasone propionate per capsule) and analyzed by Andersen cascade impaction using a Cyclohaler dry powder inhaler. The results are shown in Table 6.

TABLE 6
Respirable Dose of Dry Powder Formulations

| | | Respirable | Respirable Dose | Change in |
|---|---|---|---|---|
| | | Dose | as a Percent of | Respired Dose |
| Material | Formulation | (μg/puff) | Nominal Dose | Due to DPPC |
| Example 8a - Rep 1 | BJMB without DPPC | 168.5 | 33.70 | |
| Example 8a - Rep 2 | BJMB without DPPC | 188.1 | 37.62 | |
| Example 8a - Avg | BJMB without DPPC | 178.3 | 35.66 | |
| Example 8b - Rep 1 | BJMB with DPPC | 238.5 | 47.70 | |
| Example 8b - Rep 2 | BJMB with DPPC | 227.7 | 45.54 | |
| Example 8b - Ave | BJMB with DPPC | 233.1 | 46.22 | +30% |

The data in Table 6 show that the highest respirable dose is seen for Sample 8b, where DPPC is added to the blend prior to milling. Table 7 shows the combined effect of adding DPPC to the formulation and performing a process involving a blend of a jet milled blend.

TABLE 7
Respirable Dose of Dry Powder Formulations - Effect of Combining
DPPC in the Composition and the Blend of a Jet Milled Blend Process

| | | | | Change in |
|---|---|---|---|---|
| | | | | Respirable Dose |
| | | Respirable | Respirable | Relative to |
| | | Dose | Dose as % of | Example 7a (JMB |
| Material | Formulation | (μg/puff) | Nominal Dose | without DPPC) |
| Example 7a - Rep 1 | JMB without DPPC | 110.6 | 22.12 | |
| Example 7a - Rep 2 | JMB without DPPC | 116.4 | 23.28 | |
| Example 7a - Rep 3 | JMB without DPPC | 99.9 | 19.98 | |
| Example 7a - Avg | JMB without DPPC | 109.0 | 21.80 | |
| Example 7b - Rep 1 | JMB with DPPC | 153.4 | 30.68 | |
| Example 7b - Rep 2 | JMB with DPPC | 178.5 | 35.70 | |
| Example 7b - Rep 3 | JMB with DPPC | 162.9 | 32.58 | |
| Example 7b- Avg | JMB with DPPC | 164.9 | 32.98 | +51% |
| Example 8a - Rep 1 | BJMB without DPPC | 168.5 | 33.70 | |
| Example 8a - Rep 2 | BJMB without DPPC | 188.1 | 37.62 | |
| Example 8a - Avg | BJMB without DPPC | 178.3 | 35.66 | +64% |
| Example 8b - Rep 1 | BJMB with DPPC | 238.5 | 47.70 | |
| Example 8b - Rep 2 | BJMB with DPPC | 227.7 | 45.54 | |
| Example 8a - Avg | BJMB with DPPC | 233.1 | 46.22 | +112% |

The highest respirable dose is seen with Example 8b, which is a BJMB with DPPC in the formulation.

### Example 9

### Production of Microparticles of Fluticasone Propionate and Polymer

Microparticles containing fluticasone propionate were made as follows: 8.0 g of PLGA, 0.48 g of DPPC, and 2.2 g of fluticasone propionate were dissolved in 363.6 mL of methylene chloride. 4.0 g of ammonium bicarbonate was dissolved in 36.4 g of water. The ammonium bicarbonate solution was combined with the fluticasone priopionate/PLGA solution and emulsified using a rotor-stator homogenizer. The resulting emulsion was spray dried on a benchtop spray dryer using an air-atomizing nozzle and nitrogen as the drying gas. Spray drying conditions were as follows: 20 mL/min emulsion flow rate, 60 kg/hr drying gas rate and 20° C. outlet temperature. The product collection container was detached from the spray dryer and attached to a vacuum pump, where it was dried for 49 hours,

### Example 10

### Effect on Respirable Dose of Adding DPPC to a Blend to Make a Jet Milled Blend of Microparticles of Fluticasone Propionate and Polymer

Two different samples of fluticasone propionate formulations were prepared. Sample 10a was prepared as follows to make a jet milled blend of microparticles of fluticasone propionate and polymer without DPPC added to the blend of microparticles and lactose (the “JMB without DPPC”): Microparticles as prepared in Example 9 (0.48523 g) and 4.515 g of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar, respectively).

Sample 10b was prepared as follows to make a jet milled blend of microparticles of fluticasone propionate and polymer with DPPC added to the blend of microparticles and lactose (the “JMB with DPPC”): Microparticles as prepared in Example 9 (0.29134 g), DPPC (0.0613 g) and 2.648 g of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar).

The materials were filled into gelatin capsules (500 μg nominal fluticasone propionate per capsule) and analyzed by Andersen cascade impaction using a Cyclohaler dry powder inhaler. The results are shown in Table 8.

TABLE 8
Respirable Dose of Dry Powder Formulations

| | | Respirable | Respirable Dose | Change in |
|---|---|---|---|---|
| | | Dose | as a Percent of | Respired Dose |
| Material | Formulation | (μg/puff) | Nominal Dose | Due to DPPC |
| Example 10a - Rep 1 | JMB without DPPC | 137.8 | 27.56 | |
| Example 10a - Rep 2 | JMB without DPPC | 142.3 | 28.46 | |
| Example 10a - Avg | JMB without DPPC | 140.1 | 28.02 | |
| Example 10b - Rep 1 | JMB with DPPC | 176.6 | 35.32 | |
| Example 10b - Rep 2 | JMB with DPPC | 184.5 | 36.90 | |
| Example 10b - Avg | JMB with DPPC | 180.6 | 36.12 | +29% |

The data in Table 8 show that the highest respirable dose is seen for Sample lob, where DPPC is added to the blend prior to milling.

### Example 11

### Effect on Respirable Dose of Adding DPPC to Blend to Make a Blend of a Jet Milled Blend of Microparticles of Fluticasone Propionate and Polymer

Two different samples of fluticasone propionate formulations were prepared. Sample 11a was prepared as follows to make a blend of a jet milled blend of microparticles of fluticasone propionate and polymer without DPPC added to the blend of microparticles and lactose (the “BJMB without DPPC”): Microparticles as prepared in Example 9 (0.242 g) and 1.129 g of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar respectively). Then, the resulting milled blend (0.723 g) and 1.371 g of lactose were blended on a Turbula blender for 10 minutes at 96 rpm.

Sample 11b was prepared as follows to make a blend of a jet milled blend of microparticles of fluticasone propionate and polymer with DPPC added to the blend of microparticles and lactose (the “BJMB with DPPC”): Microparticles as prepared in Example 9 (1.2147 g), DPPC (0.2502 g) and 5.521 g of lactose were blended on a Turbula blender for 10 minutes at 96 rpm. The resulting dry blended powder was then was fed manually into a Fluid Energy Aljet spiral jet mill (injector and grinding gas pressures, 8 bar and 4 bar). Then, the resulting milled blend (6.301 g) and 4.979 g of lactose were blended on a Turbula blender for 10 minutes at 96 rpm.

The materials were filled into gelatin capsules (500 μg nominal fluticasone propionate per capsule) and analyzed by Andersen cascade impaction using a Cyclohaler dry powder inhaler. The results are shown in Table 9.

TABLE 9
Respirable Dose of Dry Powder Formulations

| | | | Respirable | Change in |
|---|---|---|---|---|
| | | Respirable | Dose as a | Respired |
| | | Dose | Percent of | Dose Due to |
| Material | Formulation | (μg/puff) | Nominal Dose | DPPC |
| Example 11a - Rep 1 | BJMB without DPPC | 182.3 | 36.46 | |
| Example 11a - Rep 2 | BJMB without DPPC | 143.3 | 28.66 | |
| Example 11 - Avg | BJMB without DPPC | 162.8 | 32.56 | |
| Example 11b - Rep 1 | BJMB with DPPC | 209.5 | 41.90 | |
| Example 11b - Rep 2 | BJMB with DPPC | 255.1 | 51.02 | |
| Example 11b - Rep 3 | BJMB with DPPC | 269.7 | 53.94 | |
| Example 11b - Rep 4 | BJMB with DPPC | 271.4 | 54.28 | |
| Example 11b - Avg | BJMB with DPPC | 251.4 | 50.28 | +54% |

The data in Table 9 show that the highest respirable dose is seen for Sample 11b, where DPPC is added to the blend prior to milling. Table 10 shows the combined effect of adding DPPC to the formulation and performing a process involving a blend of a jet milled blend.

TABLE 10
Respirable Dose of Dry Powder Formulations: Effect of Combining
DPPC in the Composition and the Blend of a Jet Milled Blend Process

| | | | Respirable | Change in |
|---|---|---|---|---|
| | | | Dose as a | Respirable Dose |
| | | Respirable | Percent of | Relative to |
| | | Dose | Nominal | Example 10a (JMB |
| Material | Formulation | (μg/puff) | Dose | without DPPC) |
| Example 10a - Rep 1 | JMB without DPPC | 137.8 | 27.56 | |
| Example 10a - Rep 2 | JMB without DPPC | 142.3 | 28.46 | |
| Example 10a - Avg | JMB without DPPC | 140.1 | 28.02 | |
| Example 10b - Rep 1 | JMB with DPPC | 176.6 | 35.32 | |
| Example 10b - Rep 2 | JMB with DPPC | 184.5 | 36.90 | |
| Example 10b - Avg | JMB with DPPC | 180.6 | 36.12 | +29% |
| Example 11a - Rep 1 | BJMB without DPPC | 182.3 | 36.46 | |
| Example 11a - Rep 2 | BJMB without DPPC | 143.3 | 28.66 | |
| Example 11a - Avg | BJMB without DPPC | 162.8 | 32.56 | +16% |
| Example 11b - Rep 1 | BJMB with DPPC | 209.5 | 41.90 | |
| Example 11b - Rep 2 | BJMB with DPPC | 255.1 | 51.02 | |
| Example 11b - Rep 3 | BJMB with DPPC | 269.7 | 53.94 | |
| Example 11b - Rep 4 | BJMB with DPPC | 271.4 | 54.28 | |
| Example 11b - Avg | BJMB with DPPC | 251.4 | 50.28 | +79% |

The highest respirable dose is seen with Example 11b, which is a BJMB with DPPC in the formulation.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

We claim:

1. A method for making a dry powder pharmaceutical formulation for pulmonary or nasal administration, comprising the steps of:
a) providing particles which comprise a pharmaceutical agent;
b) blending the particles with particles of at least one first excipient to form a first powder blend;
c) milling the first powder blend to form a milled blend which comprises microparticles or nanoparticles of the pharmaceutical agent; and
d) blending the milled blend with particles of a second excipient to form a blended dry powder blend pharmaceutical formulation suitable for pulmonary or nasal administration,
wherein the particles of second excipient are larger than the microparticles or nanoparticles in the milled blend and the second excipient is selected from the group consisting of sugars, sugar alcohols, starches, amino acids, and combinations thereof.

2. The method of claim 1, wherein the particles of at least one first excipient comprise a phospholipid.

3. The method of claim 2, wherein the phospholipid comprises dipalmitoyl phosphatidylcholine.

4. The method of claim 1, wherein the particles of the at least one first excipient comprise a material selected from the group consisting of sugars, sugar alcohols, starches, amino acids, and combinations thereof.

5. The method of claim 4, wherein a phospholipid is also blended into the first powder blend.

6. The method of claim 1, wherein the particles of second excipient comprise lactose.

7. The method of claim 1, wherein the particles of the at least one first excipient and the particles of the second excipient both comprise lactose.

8. The method of claim 1, wherein the particles of step a) are microparticles.

9. The method of claim 1, wherein the milling comprises jet milling,

10. The method of claim 1, wherein the particles of step a) are made by a spray drying process.

11. The method of claim 1, wherein the particles of step a) further comprise a shell material,

12. The method of claim 11, wherein the shell material comprises a biocompatible synthetic polymer.

13. The method of claim 1, wherein the microparticles of the milled blend which comprise the pharmaceutical agent have a volume average diameter of between 1 and 10 μm.

14. The method of claim 1, wherein the particles of the second excipient have a volume average diameter between 20 and 500 μm.

15. The method of claim 1, wherein the pharmaceutical agent comprises budesonide, fluticasone propionate, beclomethasone dipropionate, mometasone, flunisolide, triamcinolone acetonide, albuterol, formoterol, salmeterol, cromolyn sodium, ipratropium bromide, testosterone, progesterone, estradiol, enoxaprin, ondansetron, sumatriptan, sildenofil, dornase alpha, iloprost, heparin, low molecular weight heparin, desirudin, or a combination thereof.

16. A method for making a dry powder pharmaceutical formulation for pulmonary or nasal administration, comprising the steps of:
a) providing particles which comprise a pharmaceutical agent;
b) blending the particles with particles of a pre-processed excipient to form a primary blend, wherein the pre-processed excipient is prepared by
i) dissolving a bulking agent and at least one non-friable excipient in a solvent to form an excipient solution, and
ii) removing the solvent from the excipient solution to form the pre-processed excipient in dry powder form; and
c) milling the primary blend to form a milled pharmaceutical formulation blend suitable for pulmonary or nasal administration.

17. The method of claim 16, further comprising blending the milled pharmaceutical formulation blend with particles of a second excipient to form a blended dry powder blend pharmaceutical formulation suitable for pulmonary or nasal administration.

18. The method of claim 16, wherein the particles of second excipient are larger than the microparticles or nanoparticles in the milled blend and the second excipient is selected from the group consisting of sugars, sugar alcohols, starches, amino acids, and combinations thereof

19. The method of claim 16, wherein the bulking agent comprises at least one sugar, sugar alcohol, starch, amino acid, or combination thereof,

20. The method of claim 16, wherein the bulking agent is selected from the group consisting of lactose, sucrose, maltose, mannitol, sorbitol, trehalose, galactose, xylitol, erythritol, and combinations thereof.

21. The method of claim 16, wherein the non-friable excipient comprises a liquid, waxy, or non-crystalline compound.

22. The method of claim 16, wherein the non-friable excipient comprises a surfactant.

23. The method of claim 22, wherein the surfactant comprises a waxy or liquid surfactant.

24. The method of claim 16, wherein the pre-processed excipient comprises a combination of lactose and a phospholipid or fatty acid.

25. The method of claim 16, wherein the milled pharmaceutical formulation blend suitable for pulmonary or nasal administration is thermally-labile.

26. The method of claim 16, wherein the step of removing the solvent comprises spray drying, lyophilization, vacuum drying, freeze drying, or a combination thereof.

27. A method for making a dry powder blend pharmaceutical formulation, comprising the steps of:
a) providing microparticles which comprise a pharmaceutical agent;
b) blending the microparticles with particles of at least one first excipient to form a first powder blend;
c) milling the first powder blend to form a milled blend; and
d) blending the milled blend with particles of a second excipient, wherein the particles of second excipient are larger than the microparticles in the milled blend, to form a blended dry powder blend pharmaceutical formulation,
wherein the blended dry powder blend pharmaceutical formulation from step (d) exhibits an increased respirable dose as compared to a respirable dose of the microparticles of step (a), the first powder blend of step (b), or the milled blend of step (c).

28. The method of claim 27, wherein the particles of at least one first excipient comprise a phospholipid.

29. The method of claim 28, wherein the phospholipid comprises dipalmitoyl phosphatidylcholine.

30. The method of claim 27, wherein the second excipient is selected from the group consisting of sugars, sugar alcohols, starches, amino acids, phospholipids, and combinations thereof.

31. The method of claim 27, wherein the microparticles of the milled blend which comprise the pharmaceutical agent have a volume average diameter of between 1 and 10 μm.

32. The method of claim 27, wherein the particles of the second excipient have a volume average diameter between 20 and 500 μm.

33. A dry powder pharmaceutical formulation for pulmonary or nasal administration comprising a milled blend of at least one phospholipid and particles of a pharmaceutical agent.

34. The dry powder pharmaceutical formulation of claim 33, wherein the at least one phospholipid comprises dipalmitoyl phosphatidylcholine.

35. The dry powder pharmaceutical formulation of claim 33, wherein the phospholipid is combined with the particles of the pharmaceutical agent to yield a blend and the blend is then milled.

36. The dry powder pharmaceutical formulation of claim 33, wherein the phospholipid is milled and the milled phospholipid is then blended with the particles of the pharmaceutical agent.

37. A dry powder pharmaceutical formulation for pulmonary or nasal administration comprising a blend of
a milled blend of (i) microparticles which comprise a pharmaceutical agent, and (ii) excipient particles; and
particles of a sugar or sugar alcohol, which particles are larger than the microparticles or excipient particles of the milled blend,
wherein the blend exhibits an increased respirable dose as compared to a respirable dose of combinations of the microparticles, the excipient particles, and the particles of sugar or sugar alcohol, which combinations are not blend-of-milled-blend combinations.

38. The formulation of claim 37, wherein the excipient particles comprise a sugar, a sugar alcohol, a starch, an amino acid, a phospholipid, or a combination thereof.

39. The formulation of claim 37, wherein the pharmaceutical agent has a solubility in water of less than 10 mg/mL at 25° C.

40. The formulation of claim 37, wherein the pharmaceutical agent comprises budesonide, fluticasone propionate, beclomethasone dipropionate, mometasone, flunisolide, triamcinolone acetonide, albuterol, formoterol, salmeterol, cromolyn sodium, ipratropium bromide, testosterone, progesterone, estradiol, enoxaprin, ondansetron, sumatriptan, sildenofil, dornase alpha, iloprost, heparin, low molecular weight heparin, desirudin, or a combination thereof.

41. The formulation of claim 37, wherein the sugar or sugar alcohol comprises lactose, sucrose, maltose, mannitol, sorbitol, trehalose, galactose, xylitol, erythritol, or a combination thereof.

42. The formulation of claim 37, wherein the microparticles which comprise pharmaceutical agent have a volume average diameter of less than 10 μm.

43. The formulation of claim 37, wherein the microparticles which comprise pharmaceutical agent have a volume average diameter of less than 5 μm.

44. The formulation of claim 37, wherein the excipient particles and the particles of the sugar or sugar alcohol both comprise lactose.

45. The formulation of claim 37, wherein the particles of step (a) further comprise a shell material.

46. The formulation of claim 45, wherein the shell material comprises a biocompatible synthetic polymer.

47. The formulation of claim 37, wherein the particles of the sugar or sugar alcohol have a volume average diameter between 20 and 500 μm.

48. The formulation of claim 37, wherein the blend is made by a process comprising:
a) blending particles which comprise a pharmaceutical agent with particles of at least one first excipient to form a first powder blend;
b) milling the first powder blend to form a milled blend which comprises the microparticles of the pharmaceutical agent; and
c) blending the milled blend with the particles of a sugar or sugar alcohol to form the blend.

49. The formulation of claim 37, wherein the milled blend is made by a process comprising:
a) providing particles which comprise a pharmaceutical agent;
b) blending the particles with particles of a pre-processed excipient to form a primary blend, wherein the pre-processed excipient is prepared by
i) dissolving a bulking agent and at least one non-friable excipient in a solvent to form an excipient solution, and
ii) removing the solvent from the excipient solution to form the pre-processed excipient in dry powder form; and
c) milling the primary blend to form the milled blend.

50. A dry powder pharmaceutical formulation for pulmonary or nasal administration comprising:
a milled blend of (i) microparticles which comprise a pharmaceutical agent, (ii) at least one phospholipid, and (iii) tertiary excipient particles; and
particles of a sugar or sugar alcohol, which particles are blended with the milled blend and are larger than the microparticles or excipient particles of the milled blend.

51. The formulation of claim 50, wherein the at least one phospholipid comprises dipalmitoyl phosphatidylcholine.

52. The formulation of claim 50, wherein the tertiary excipient particles comprise a sugar, a sugar alcohol, a starch, an amino acid, or a combination thereof

53. The formulation of claim 50, wherein the sugar or sugar alcohol comprises lactose, sucrose. maltose, mannitol, sorbitol, trehalose, galactose, xylitol, erythritol, or a combination thereof.
